(19) United States
(12) Patent Application Publication
(10) Pub. No.: US 2010/0305397 A1
(43) Pub. Date: Dec. 2, 2010

(54) HYDRAULIC-MECHANICAL GASTRIC BAND

(75) Inventors: Janel A. Birk, Oxnard, CA (US); Pierre Fridez, Froideville (CH); Tiago Bertolote, Geneva (CH)

Correspondence Address: ALLERGAN, INC., 2525 DUPONT DRIVE, T2-7H, IRVINE, CA, 92612-1599, US

(73) Assignee: Allergan Medical Sarl, Lausanne (CH)

(21) Appl. No.: 12/816,310

(22) Filed: Jun. 15, 2010

Related U.S. Application Data
(63) Continuation-in-part of application No. 12/574,640, filed on Oct. 6, 2009.
(60) Provisional application No. 61/103,153, filed on Oct. 6, 2008.

(51) Int. Cl. A61F 2/00

(52) U.S. Cl. 600/37

(57) ABSTRACT

An implantable banding system for treating obesity is disclosed. The implantable banding system includes a telemetric control unit, a gastric band having at least one inner fluid compartment and an outer mechanical adjustment mechanism, the at least one inner fluid compartment being filled with a fixed volume of fluid, and the outer mechanical adjustment mechanism comprising a device configured to adjust the gastric band through a variety of diameters, an implant circuit coupled to the device and configured to receive an adjustment signal to control the operations of the device, and a sensor positioned within the at least one inner fluid compartment, configured to monitor a parameter of the fixed volume of fluid, generate an adjustment signal based on the parameter and one or more parameter control limits, and automatically activate the device based on the adjustment signal or transmit the adjustment signal to the telemetric control unit.

### RELATED APPLICATIONS

This application is a continuation in part of, and claims priority to and the benefit of, U.S. patent application Ser. No. 12/574,640, entitled “MECHANICAL GASTRIC BAND WITH CUSHIONS” filed on Oct. 6, 2009, which claims priority to and the benefit of U.S. Provisional Patent Application No. 61/103,153, filed on Oct. 6, 2008, the entire disclosures of which are incorporated herein by reference.

### FIELD

This invention relates to surgical devices for regulating or controlling an organ or a duct, for example, a gastric banding system.

### BACKGROUND

Obesity is well recognized as a serious health problem, and is associated with numerous health complications, ranging from non-fatal conditions to life threatening chronic diseases. According to the World Health Organization, debilitating health problems associated with obesity include respiratory difficulties, chronic musculoskeletal problems, skin problems and infertility. Life-threatening problems fall into four main areas: cardiovascular disease problems; conditions associated with insulin resistance such as type 2 diabetes; certain types of cancers, especially the hormonally related and large bowel cancers; and gallbladder disease. Beyond these physiological problems, obesity has also psychological consequences, ranging from lowered self-esteem to clinical depression.

Surgical intervention is sometimes indicated for people suffering from the effects of obesity. Such intervention not only mitigates the myriad health problems arising from being overweight, but may reduce the risk of early death of the patient. Left untreated, morbid obesity may reduce a patient's life expectancy by ten to fifteen years.

### SUMMARY

An implantable banding system for treating obesity is disclosed. The implantable banding system includes a telemetric control unit, a gastric band having at least one inner fluid compartment and an outer mechanical adjustment mechanism, the at least one inner fluid compartment being filled with a fixed volume of fluid, and the outer mechanical adjustment mechanism comprising a device configured to adjust the gastric band through a variety of diameters, an implant circuit coupled to the device and configured to receive an adjustment signal to control the operations of the device, and a sensor positioned within the at least one inner fluid compartment, configured to monitor a parameter of the fixed volume of fluid, generate an adjustment signal based on the parameter and one or more parameter control limits, and automatically activate the device based on the adjustment signal or transmit the adjustment signal to the telemetric control unit.

A system for regulating an organ or duct, for example, the functioning of an organ or duct, is provided. The system generally comprises an implantable band having a first end and a second end, a distal region and a proximal region, and a connector configured to couple the first end with the second end such that the band is formable into a loop configuration. The band is structured to circumscribe, or at least partially circumscribe, an organ or duct, for example, a stomach. The system further comprises a mechanism for enabling adjustment of an inner circumference of the loop configuration to effect constriction of the organ or duct.

For the sake of simplicity, and in no way intended to limit the scope of the invention, the “organ or duct” will hereinafter typically be referred to as a “stomach” and the system will be described as a gastric band system. The band is structured to circumscribe an upper portion of a stomach to form a stoma that controls the intake of food to the stomach. It is to be appreciated that although the invention is hereinafter typically described as pertaining to a gastric band system for application to a stomach, for example, for obesity treatment, the system, with appropriate modification thereto, can be used for regulating or controlling any organ or duct that would benefit from application of the present system thereto.

Once the band is implanted about the stomach, the size of an inner diameter of the band can be adjusted to provide the desired degree of restriction. Techniques for determining appropriate adjustment of gastric bands, timing and amount of adjustments, are known in the art and therefore will not be described in great detail herein.

Advantageously, in a broad aspect of the invention, the system may be structured to substantially prevent or at least reduce the occurrence of pinching of the body tissues, for example, the tissues of the stomach, during constriction or tightening of the band.

For example, in a specific embodiment, the system further comprises a contact region located between the first end and the second end of the band which is structured and functions to progressively move tissue, for example stomach tissue, during tightening of the band, without entrapping the tissue.

The contact region may comprise a plurality of first segments and a plurality of second segments arranged in a generally alternating manner along the proximal (e.g. stomach-facing) region of the band. The first segments may comprise relatively wide, substantially incompressible cushion segments, and the second segments may comprise relatively thin, elastic tension segments. During constriction of the band, adjacent incompressible cushion segments form a progressively narrowing angle, for example, a substantially V-shaped surface. A tension segment is located between the adjacent cushion segments and forms the vertex of the angle or V.

In some embodiments, the cushion segments and tension segments form an inner circumference of the loop configuration having a generally star-shape, defined by the contact region. Deformation of the star-shape during adjustment substantially or entirely prevents pinching of tissues, as the cushion segments roll forward towards one another without gaps there-between thus pushing the tissue inwardly.

More specifically, in some embodiments, the contact region defines alternating convex stomach-facing surfaces and concave stomach-facing surfaces. The convex organ facing surfaces may be defined by the cushion segments and the convex organ facing surfaces are defined by the tension segments located between adjacent cushion segments. During constriction of the band, the convex organ-facing surfaces may maintain their shape while folding at the tension segments inwardly toward one another. This mechanism and structure causes the tissues of the stomach to be pushed outwardly from the band constriction without the tissues becoming entrapped and/or pinched by the contact region.

In addition, the structure of the contact region, including cushion segments and tension segments, may be advantageously structured to maintain mechanical stability of the band. For example, the tension segments provide a means for maintaining positioning of the cushion segments and by substantially preventing the contact region of the band from creasing, folding or rolling out of position while the band is implanted in the body around the duct or organ, for example, the stomach.

In some embodiments, the contact region comprises a membrane, for example, a somewhat tubular-shaped elastic membrane encompassing, secured to or defining the cushion segments. In one embodiment, portions of the membrane may form the tension segments between adjacent cushion segments.

In one embodiment, the cushion segments are formed of individual incompressible molded elements in contact with or spaced apart from one another, and affixed to the membrane. The cushion segments may be spaced apart by portions of the elastic membrane which are stretched under tension.

The cushion segments may be located on an internal surface of the membrane or alternatively may be located on an external surface of the membrane. In one embodiment, the cushion segments are located on an external surface of the membrane and are overmolded to the membrane.

In another feature of the invention, the membrane may include structure, for example, corrugations or indentations, for facilitating expansion of the membrane during adjustment of the loop. For example, such corrugations can be located and structured to minimize the force required to elongate or stretch the membrane in the radial direction during tightening of the band. The corrugated surfaces of the membrane reduce membrane deformation energy by allowing the membrane to unfold rather than stretch during adjustment.

The mechanism for enabling adjustment may comprise an electronic interface, for example, an implantable electronic interface connected to the band, and a control, for example an external control unit, capable of communicating with the interface to regulate the constriction of the band about the organ or the duct.

In one embodiment, the present invention is an implantable banding system for treating obesity, the implantable banding system including a gastric band having at least one inner fluid compartment and an outer mechanical adjustment mechanism, the at least one inner fluid compartment being filled with a volume of fluid, and the outer mechanical adjustment mechanism comprising a device configured to adjust the gastric band through a variety of diameters.

In another embodiment, the present invention is an implantable banding system for treating obesity, the implantable banding system including a telemetric control unit, a gastric band having at least one inner fluid compartment and an outer mechanical adjustment mechanism, the at least one inner fluid compartment being filled with a fixed volume of fluid, and the outer mechanical adjustment mechanism comprising a device configured to adjust the gastric band through a variety of diameters, a sensor positioned within the at least one inner fluid compartment, configured to monitor a parameter of the fixed volume of fluid, and generate data based on the parameter to be monitored, an implant circuit coupled to the device and configured to analyze the data from the sensor, and control operations of the device based on the data from the sensor including automatically activating the device based on the data from the sensor or transmit the data from the sensor to the telemetric control unit.

In yet another embodiment, the present invention is a method for treating obesity including using a gastric band having at least one inner fluid compartment and an outer mechanical adjustment mechanism, the at least one inner fluid compartment being filled with a volume of fluid, and the outer mechanical adjustment mechanism comprising a device configured to adjust the gastric band through a variety of diameters.

These and other features of the present invention may be more clearly understood and appreciated upon consideration of the following Detailed Description and the accompanying Drawings.

### BRIEF DESCRIPTION OF THE DRAWINGS

FIG. 1 shows a system including a band having a contact region, an interface having an antenna/controller pod, and an external control in accordance with an exemplary embodiment of the present invention.

FIG. 2 shows a perspective, cutaway view of the contact region shown in FIG. 1 in accordance with an exemplary embodiment of the present invention.

FIG. 3 shows a perspective view of the contact region shown in FIG. 1 in accordance with an exemplary embodiment of the present invention.

FIG. 3A shows a cross-sectional view of the contact region taken along lines 3A-3A of FIG. 3 in accordance with an exemplary embodiment of the present invention.

FIG. 4A shows an elevation view of the contact region shown in FIG. 1 in accordance with an exemplary embodiment of the present invention.

FIG. 4B shows an elevation view of an alternative contact region in accordance with an exemplary embodiment of the present invention.

FIG. 4C shows a perspective view of the alternative contact region shown in FIG. 4B in accordance with an exemplary embodiment of the present invention.

FIG. 5A shows a cross-sectional view of the band shown in FIG. 1 in accordance with an exemplary embodiment of the present invention.

FIG. 5B shows a cross-sectional view of the band taken along lines 5B-5B of FIG. 5A in accordance with an exemplary embodiment of the present invention.

FIG. 5C shows a perspective, cutaway view of the band in a fully open position in accordance with an exemplary embodiment of the present invention.

FIG. 5D shows a perspective, cutaway view of the band in a constricted position in accordance with an exemplary embodiment of the present invention.

FIGS. 5E and 5F are schematic representations of an amplified adjustment feature in accordance with an exemplary embodiment of the present invention.

FIGS. 5G and 5H are simplified schematic representations of another embodiment of the invention in accordance with an exemplary embodiment of the present invention.

FIGS. 6A through 6C show plan views of the band at different levels of constriction in accordance with an exemplary embodiment of the present invention.

FIG. 7 is a partial perspective view of a screw thread portion of a tension element useful in the band of the system in accordance with an exemplary embodiment of the present invention.

FIG. 8 is a perspective view of the entire tension element shown in FIG. 7 in accordance with an exemplary embodiment of the present invention.

FIG. 9 is a perspective view of the entire tension element of FIG. 8 coupled to a rigid distal peripheral portion in the band of the system in accordance with an exemplary embodiment of the present invention.

FIG. 10 is a perspective view of the band of the system in a straightened configuration and located within a trocar to facilitate implantation in accordance with an exemplary embodiment of the present invention.

FIG. 11 is a cross-sectional view of an actuator housing on an end of the band in accordance with an exemplary embodiment of the present invention.

FIG. 12 is a perspective view of an actuator in the housing shown in FIG. 11 in accordance with an exemplary embodiment of the present invention.

FIG. 13 is a perspective view of the tension element engaged with the actuator shown in FIG. 12 in accordance with an exemplary embodiment of the present invention.

FIG. 14 is a cross-sectional view depicting the construction of the actuator shown in FIG. 12 in accordance with an exemplary embodiment of the present invention.

FIG. 15 is a cross-sectional view depicting the construction of a reference position switch useful in the system in accordance with an exemplary embodiment of the present invention.

FIGS. 16A and 16B are perspective views illustrating a clip used to close the band of the system in accordance with an exemplary embodiment of the present invention.

FIG. 17 is a perspective view of the antennae/controller pod of the system shown in FIG. 1 in accordance with an exemplary embodiment of the present invention.

FIG. 18 is a cut-away view of the interior of the implantable antenna/controller pod in accordance with an exemplary embodiment of the present invention.

FIG. 19 is a schematic view of telemetric power and control circuitry useful in the system in accordance with an exemplary embodiment of the present invention.

FIG. 20 is a view of a signal strength indicator portion of the control shown in FIG. 1 in accordance with an exemplary embodiment of the present invention.

FIG. 21 is a schematic diagram illustrating placement of the implantable portion of the system in accordance with an exemplary embodiment of the present invention.

FIGS. 22A-22H are each a view illustrating steps in a method of laparoscopically implanting the system in accordance with an exemplary embodiment of the present invention.

FIG. 23 is a perspective view of a contact region including a membrane and overmolded incompressible cushions of a gastric band in accordance with an exemplary embodiment of the present invention.

FIGS. 24 and 25 are cross-sectional views of the contact region shown in FIG. 23 taken along line 24-24 and line 25-25, respectively, in accordance with an exemplary embodiment of the present invention.

FIGS. 26 and 26A show dilated cushion segments and tension segments forming an inner circumference of the loop configuration having a generally star-shape in accordance with an exemplary embodiment of the present invention.

FIGS. 27 and 27A show constricted cushion segments and tension segments forming an inner circumference of the loop configuration having a generally star-shape in accordance with an exemplary embodiment of the present invention.

FIG. 28 illustrates an implantable banding system in accordance with an exemplary embodiment of the present invention.

FIGS. 29A and 29B illustrate exemplary fixed-volume compartments in accordance with an exemplary embodiment of the present invention.

FIG. 30 illustrates an implantable banding system further comprising an override mechanism in accordance with an exemplary embodiment of the present invention.

FIG. 31 illustrates a working embodiment of an implantable banding system in accordance with an exemplary embodiment of the present invention.

FIG. 32 illustrates a pressure chart according to an embodiment of the present invention.

FIG. 33 depicts a sectional view of a gastric band according to an embodiment of the present invention.

FIG. 34 depicts a sectional view of a gastric band according to an embodiment of the present invention.

### DETAILED DESCRIPTION

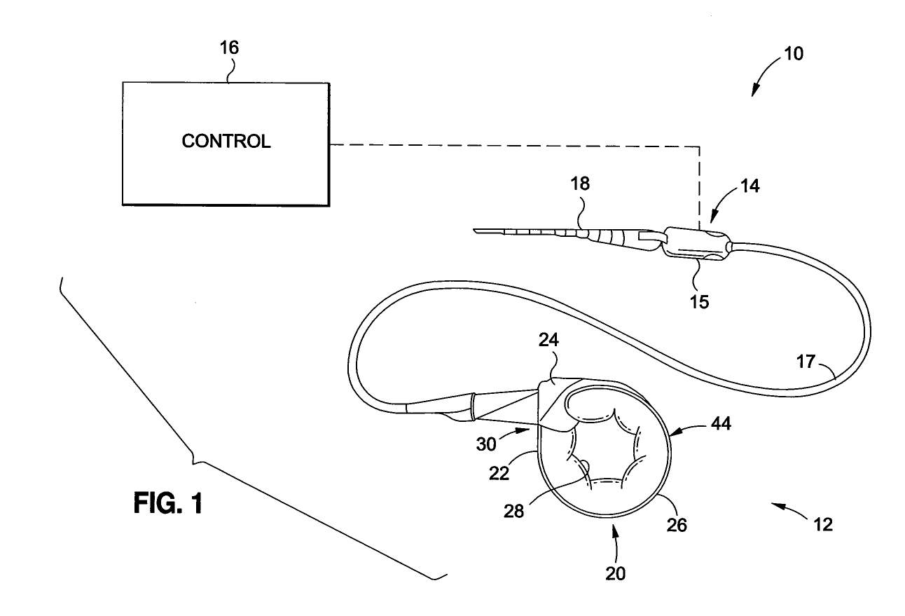

Turning now to FIG. 1, an embodiment of a system of the present invention is generally shown at 10. In one embodiment of the present invention, the system 10 is useful for regulating the functioning of an organ or duct (not shown), for example, a stomach or a stoma of the stomach. In one embodiment, the system 10 is a gastric banding system useful in the treatment of obesity and/or obesity related diseases.

It is to be understood that although much of the following description is generally directed to gastric banding systems of the invention, the present invention is in no way limited thereto. Other embodiments of the invention may be applied to regulate the functioning of other body organs or ducts, such as in the treatment of gastro-esophageal reflux disease, urinary or fecal incontinence, colostomy, or to regulate blood flow.

In this exemplary embodiment, the system 10 generally comprises an implantable portion 12 including an adjustable band 20, an interface 14 including an antenna/controller pod 15, and a control 16 in communication, for example, telemetric communication, with the pod 15. The pod 15 may be connected to the band 20 by means of an antenna cable 17 and may include a removable tab 18 for facilitating laparoscopic positioning thereof.

Laparoscopically implanted gastric bands and their use in the treatment of obesity are now well known. Generally, in accordance with the present invention, the band 20 is structured to be implantable in a patient, for example, laparoscopically implantable, around an upper region of the patient's stomach, thereby forming a stoma that restricts food intake and provides feelings of satiety. The inner diameter of the band 20 is adjustable in vivo in order to enable a physician or patient to achieve most desirable stoma size, and the best clinical results.

The band 20 includes a first end 22 and a second end 24, a distal region 26 and a proximal region 28, and a connector 30 configured to couple the first end 22 with the second end 24 of the band 20 such that the band 20 is formable into a loop configuration, as shown.

When the band 20 is formed into the loop configuration, the proximal region 28 forms an inner circumferential surface which at least partially circumscribes and contacts the organ or duct, for example, the stomach portion, to be regulated or controlled.

Generally, by loosening or tightening the band 20 about the stomach portion, regulation and/or functioning of the stomach can be controlled or adjusted. When not connected at the first and second ends 22, 24, the band 20 can be temporarily straightened in order to facilitate surgical implantation, for example, via laparoscopic techniques.

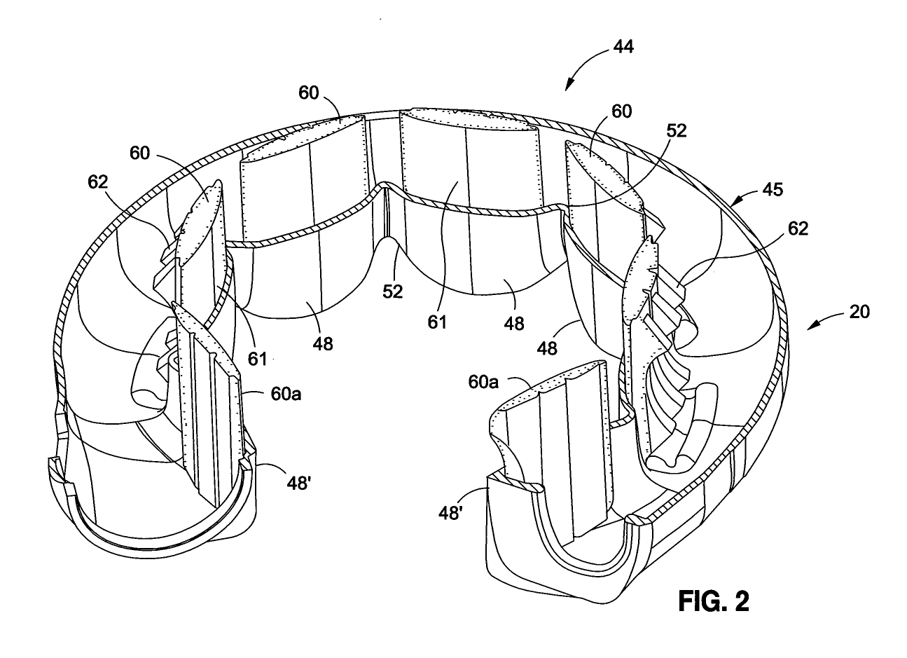

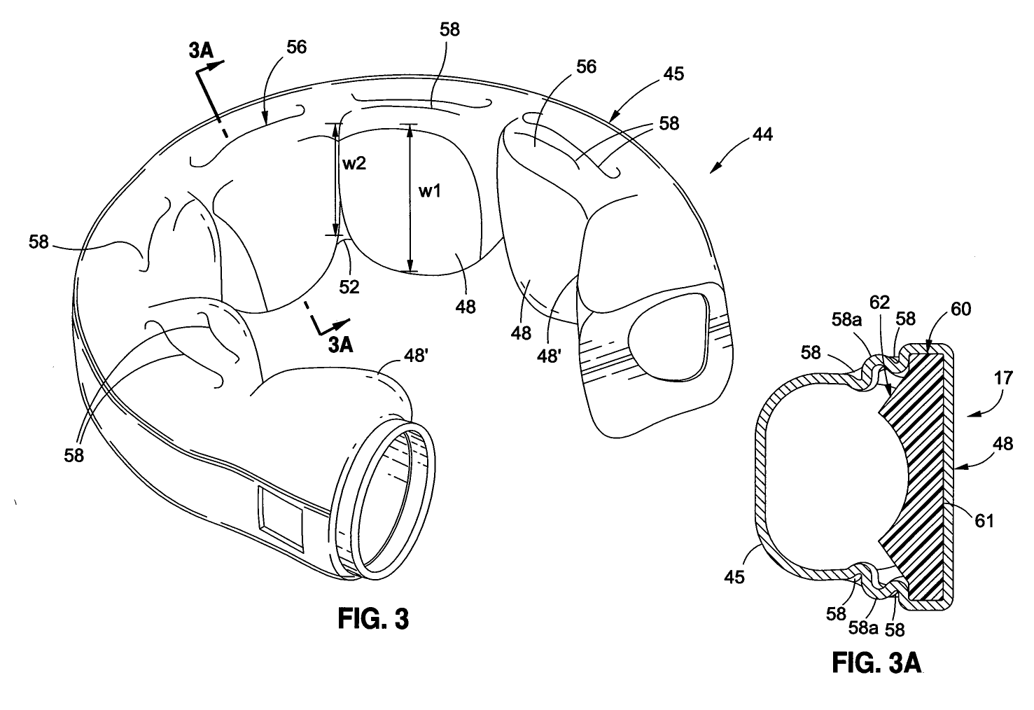

The system 10 further comprises a contact region 44 disposed between the first and the second ends 22, 24 of the band 20. Turning now to FIGS. 2 and 3, the contact region 44 may comprise, at least in part, an elastic component made of, for example, a molded silicone elastomer. The elastic component comprises a membrane 45 having a generally tubular form which covers or encases the internal mechanisms of the band 20, for example, the gastric band tightening mechanisms such as those to be described hereinafter. The membrane 45, when at rest, may have an arcuate or C-shaped form.

As shown in FIG. 2, the contact region 44 comprises first segments 48 and second segments 52 arranged in a generally alternating manner. The first segments 48 may be defined by generally planar and/or convex stomach-facing surfaces, i.e., proximal surfaces, of the contact region 44. The second segments 52 may be defined by generally concave exterior surfaces generally forming indentations between the first segments 48.

In some embodiments, the first segments 48 comprise cushions 60. The cushions 60 are spaced apart from one another by the second segments 52. The cushions 60 may be made of non-compressible material, for example, a silicone elastomer.

In one embodiment of the present invention, a suitable incompressible material making up the cushions 60 is a moldable material that has substantially constant density throughout and maintains its volume when deformed. The volume of incompressible materials cannot be reduced more than a nominal amount (e.g., about 5%) when subjected to static compression, or external pressure. The cushions 60 may be a soft silicone material that is a deformable, resilient solid or a gel. In other embodiments, the cushions 60 may be filled with non-compressible liquid, for example, a saline solution.

The cushions 60 may be made of a material that has a different durometer, for example, is softer than the material forming the membrane 45. In a specific embodiment, the cushions 60 comprise a soft, molded silicone elastomer material having a hardness of about 5 Shore A. The membrane 45 comprises a soft molded silicone elastomer material having a hardness of about 30 Shore A.

In one embodiment, the cushions 60 may be structured to provide form, definition, support and/or structural integrity to the first segments 48. The second segments 52 may be portions of the membrane 45 which are stretched under tension. The second segments 52 may be structured to provide stability to the contact region 44 and to maintain positioning, for example, circumferential positioning, of the cushions 60 during use of the system 10.

Turning now to FIG. 3, the first segments 48 may have a first axial width W1, and the second segments 52 may have a second axial width W2 which is less than the first axial width W1.

In the shown embodiment of the present invention, the contact region 44 includes seven first segments 48 (including 48′), each first segment being generally equally spaced apart by intermediate second segments 52. In other embodiments of the present invention, the contact region 44 includes at least three first segments, at least four first segments, at least five first segments, or at least six first segments. In other embodiments of the present invention, the contact region 44 includes more than seven first segments, for example, up to ten first segments or more.

In another embodiment of the present invention, the membrane 45 may be structured to facilitate expansion in a radial direction during adjustment of the inner circumference of the band 20. For example, turning now to FIG. 3, the membrane 45 may include radially expandable surfaces 56. For example, the membrane 45 includes one or more corrugations 58.

In the shown embodiment, the corrugations 58 are generally aligned with the cushions 60. As shown in FIG. 3A, the corrugations 58 may be defined by convolutions 58a defined in an upper surface and/or a lower surface of the membrane 45. The corrugations 58 may be placed to minimize the force required by the actuating mechanism to elongate the membrane 45 in the radial direction. Rather than requiring excessive stretching of the membrane 45, the membrane 45 unfolds during adjustment.

In the shown embodiment, certain first segments 48 include corrugations 58 and other first segments (e.g. first segments 48′) do not include corrugations. For example, intermediate first segments 48 include corrugations 58 and terminal first segments 48′ do not include corrugations.

The presently described and shown corrugated structure of the contact region 44 may function to facilitate controlled expansion and/or contraction of the first segments 48, for example, during adjustment of the inner circumference of the band. In some embodiments of the invention, the corrugated surfaces 56 function, at least in part, to decrease the level of force required to adjust the inner circumference of the loop.

In some embodiments, the contact region 44 includes first cushions 60 and second cushions 60a which are configured somewhat differently than first cushions 60 (see FIG. 2). In the shown embodiment, first cushions 60 are located on intermediate first segments 48 and second cushions 60a are located on terminal first segments 48′ (i.e., those first segments located at the extremities of the contact region 44).

More specifically, in the embodiment shown in FIG. 2, each first cushion 60 includes a substantially planar or convex face 61 and at least one or more distal projections 62. For example, each cushion 60 includes three longitudinal, arcuate projections 62 as shown. A cross-sectional view of first cushion 60 having these features is also shown in FIG. 3A.

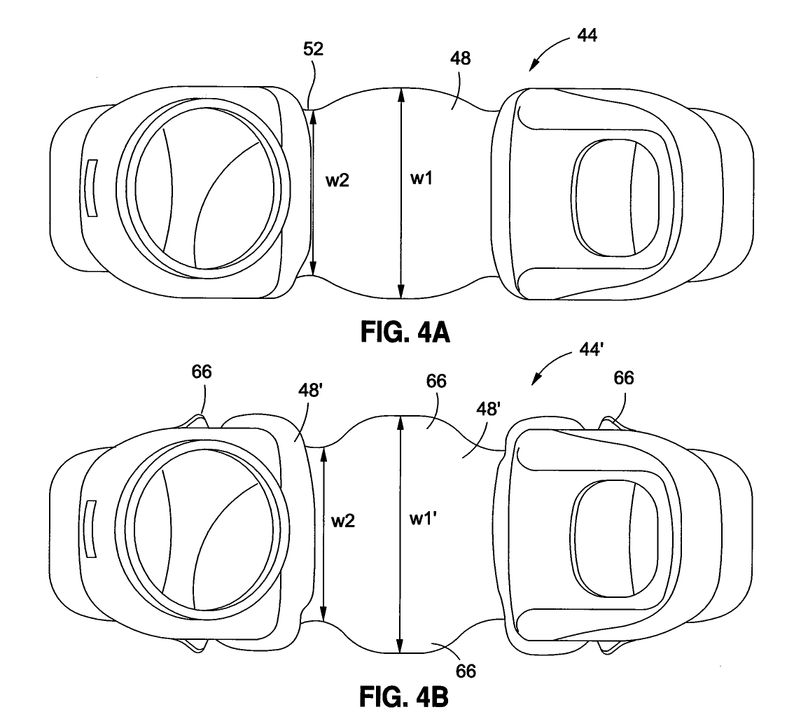

FIG. 4A shows an elevation view of the contact region 44 (cushions not shown) in order to illustrate width W1 of first segment 48 relative to width W2 of second segment 52 of contact region 44. In an exemplary embodiment of the present invention, W1 is about 17 mm and W2 is about 13 mm.

FIG. 4B shows an elevation view of an alternative contact region 44′ in accordance with an exemplary embodiment of the present invention. The contact region 44′ is identical to the contact region 44 shown in FIG. 4A, with a primary difference being that the first segment width W1′ of contact region 44′ is greater than first segment width W1 of contact region 44. That is, W1′>W1. The additional width of the first segment width W1′ is provided by upper and lower protuberances 66 on the first segments 48′. In an exemplary embodiment, W1′ is about 19 mm and W2 is about 13 mm. FIG. 4C shows a perspective view of the contact region 44′ having first segments 48′ with protuberances 66.

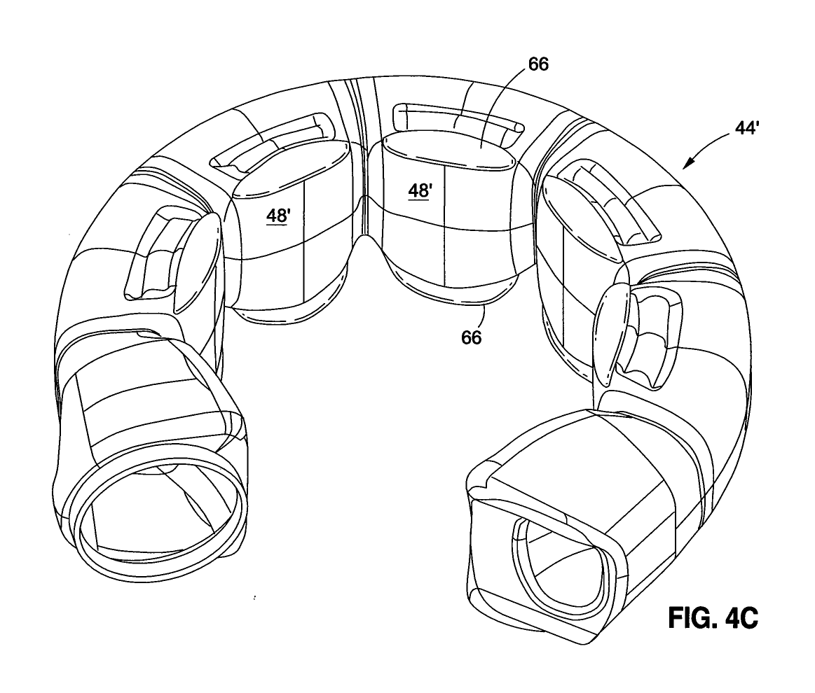

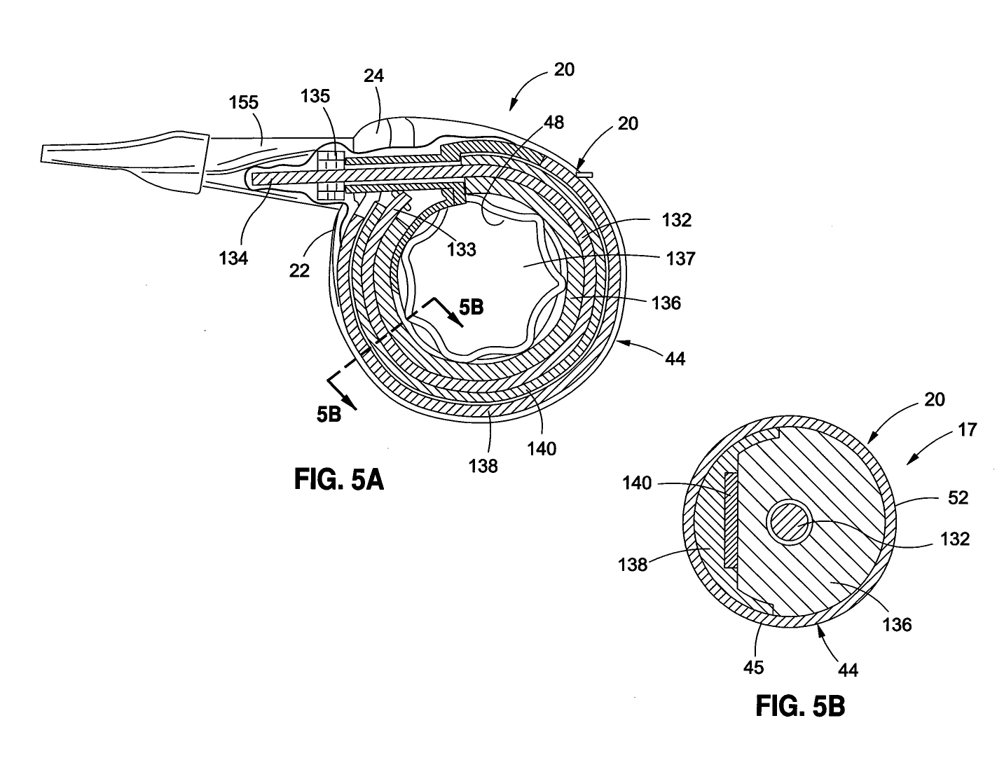

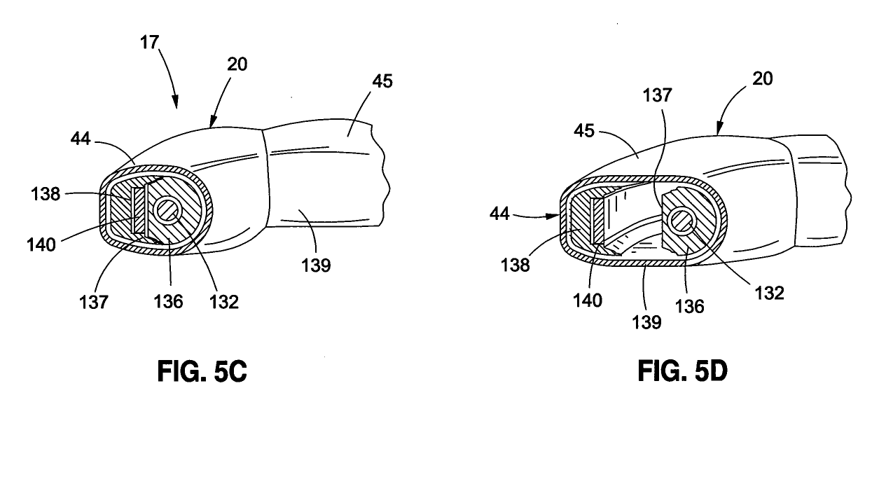

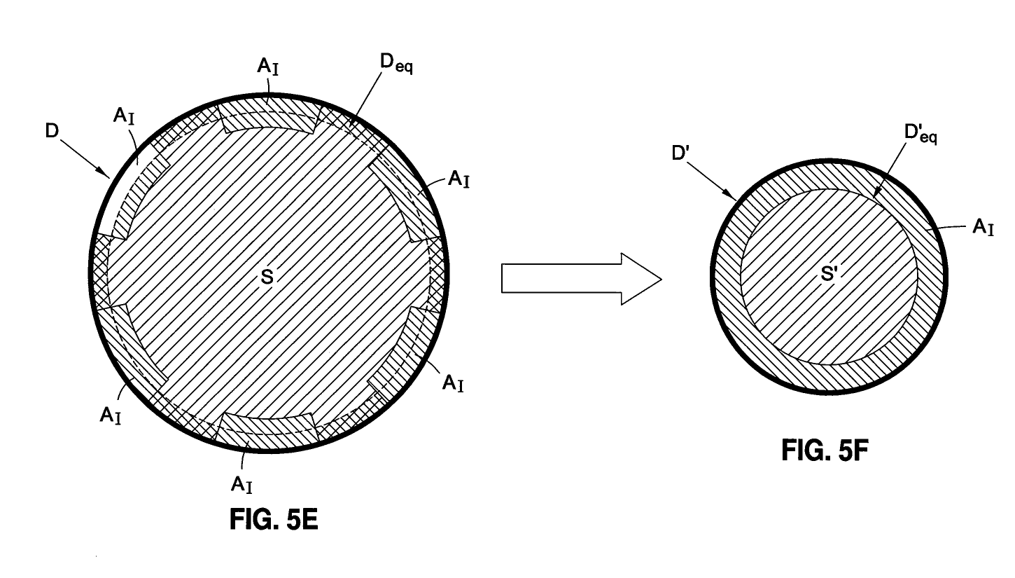

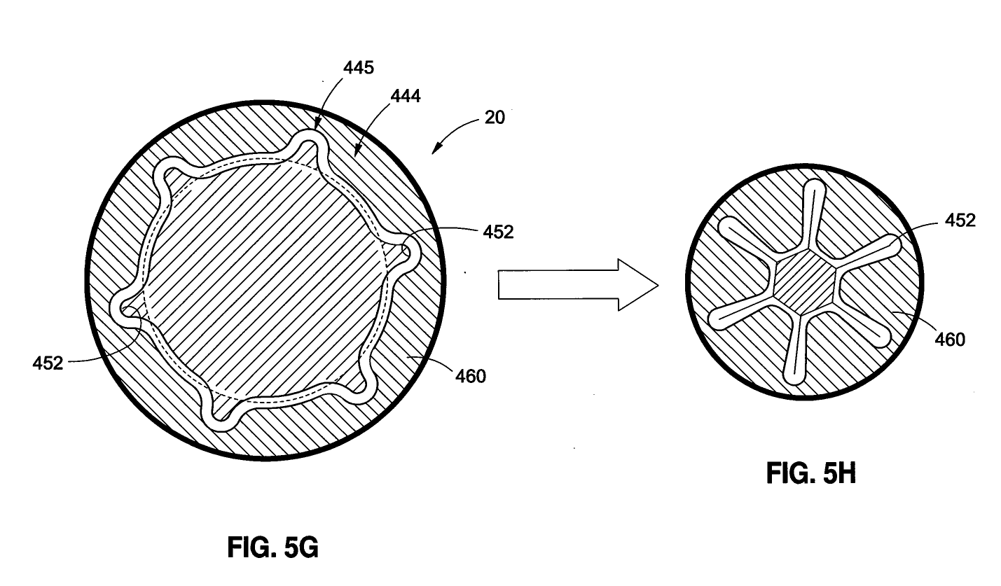

Turning now to FIGS. 5A-5D, an exemplary inner mechanism of the band 20 which enables adjustment of the inner circumference of the loop configuration will now be described. The band 20 may comprise a flexible tension element 132 having a fixed end 133 mounted to a first end 22 of the band 20 and another end 134 that is coupled to an actuator 135 at a second end 24 of the band 20. The flexible tension element 132 is slidingly disposed within a substantially cylindrical tube of axially compressible material 136. When the flexible tension element 132 is pulled through the actuator 135, a compressible material 136 is compressed and the diameter of loop opening 137 is reduced.

Turning now specifically to FIGS. 5C and 5D, a compressible material 136 may be surrounded on a distal face 137 thereof with a flexible, relatively sturdy elastomeric material, such as a silicone element 138. Both the compressible material 136 and the silicone element 138 are enclosed within the membrane 45 of the contact region 44.

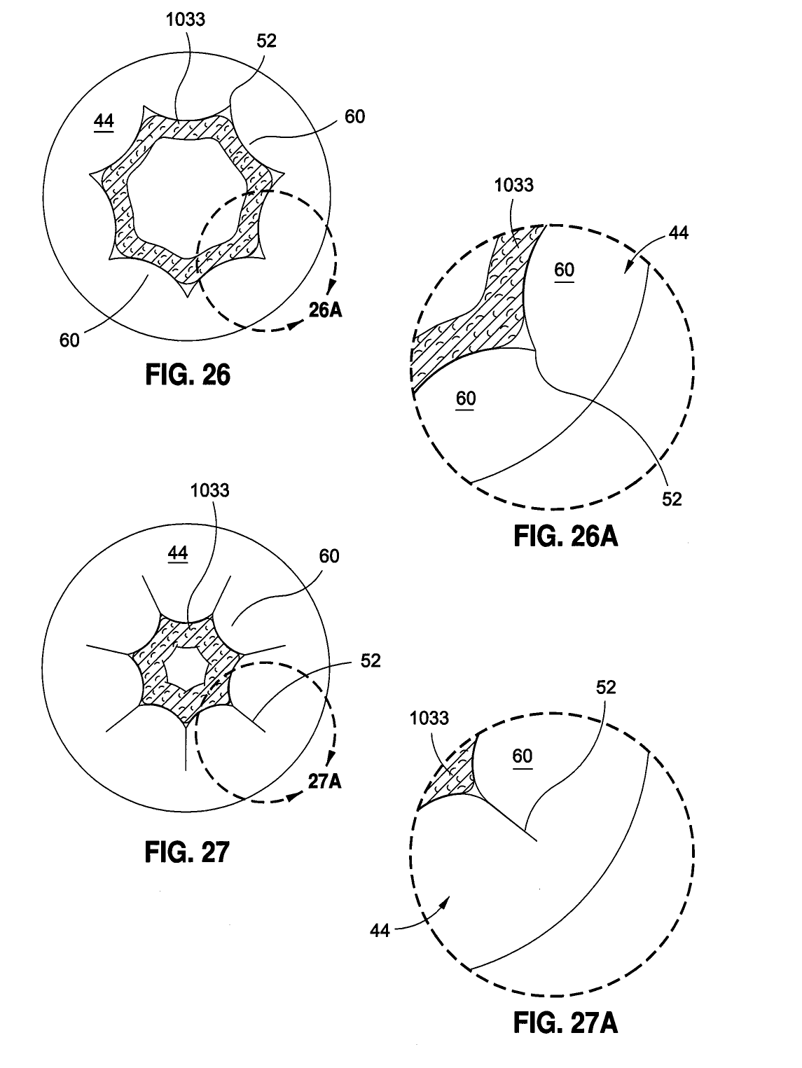

In one embodiment of the present invention, the band 20 may be structured to provide an amplified adjustment feature. This concept is illustrated in FIGS. 5E and 5F and in FIGS. 26 thru 27A.

The incompressible cushions 60 provide enhanced and more efficient control of adjustment of the inner diameter of the band 20. FIGS. 5E and 5F are schematic representations of the cross-section of the band 20 in the open configuration and constricted configuration, respectively. The outer diameter D represents the outer diameter of an axially adjustable portion of the band 20. Areas of the individual cushions 60 are represented by areas A_{I} in FIG. 5E (open configuration). The total area occupied by the individual cushions is represented as annular area A_{T} in FIG. 5F (constricted configuration). The surface S represents the available lumen around the stomach (or other organ or duct being controlled or regulated) and diameter Deq represents an equivalent diameter, that is, the diameter of a circle having the same surface area as S.

When the loop is constricted from the fully open state, diameter D (FIG. 5E) becomes D′ (FIG. 5F), the surface S becomes S′ and the equivalent diameter Deq becomes D′ eq. Because the cushions occupying A_{I} are incompressible, the total surface area A_{T} occupied by the cushions does not change. The equivalent diameter Deq decreases more rapidly than the diameter D.

For example, D=29 mm in a fully open position and a total surface of the incompressible cushions A_{T} equal to about 120 square mm: S=540.52 sq mm and Deq=26.2 mm. When in the fully closed position, D′=19 mm: S′=163.53 sq mm, and D′ eq=14.4. Thus D-D′=10 mm, and Deq-D′ eq=11.8 mm, which provides an “amplification factor” of about 1.18. Thus, by changing the values of D, D′ and A_{T}, the amplification factor can be controlled.

The substantially incompressible cushion segments allow a relative restriction of the lumen during adjustment greater than without substantially incompressible cushion segments. That greater relative restriction arises from the fact that the cross-section of the substantially incompressible cushion segments remains constant during adjustment, whereas the area of the lumen decreases during closure, so that the ratio (cushion cross-section)/(lumen) increases. Accordingly, the substantially incompressible cushion segment effect on lumen restriction increases during closure.

FIGS. 5G and 5H show a simplified schematic representation in which the contact region 444 comprises an elastic membrane 445 and a single continuous, incompressible cushion segment 460 instead of the individual, separate cushion segments 60 shown in FIG. 2. Other than cushion segment 460 being a single substantially continuous cushion segment rather than a plurality of individual separate cushion segments 60, the band 20 (FIG. 5G) may be identical to the band 20 (FIG. 2). The continuous cushion segment 460 is configured or shaped to accommodate tension segments 452 of the membrane 445. For example, the continuous cushion segment 460 has a variable thickness, with the thickest regions functioning similarly to incompressible cushion regions 60 described elsewhere herein. FIG. 5H shows bending of tension regions 452 and deformation of incompressible cushions 460 during the constriction of the loop.

Turning back to FIG. 5A, the band 20 may further comprise member 140 of a relatively rigid material. By its structural rigidity, member 140 imposes a generally circular arc shape for the entirety of the band 20. In some embodiments of the present invention, rigidity of the band 140 functions to prevent the exterior diameter of the band 20 from changing during adjustment of the internal diameter of the loop.

Generally, an increase or reduction of the length of the tension element 132 results in reversible radial displacement at the internal periphery of the band 20. This in turn translates into a variation of internal diameter of the loop from a fully open diameter to a fully closed diameter.

In various embodiments of the present invention, the diameter of the opening 137 formed by the band 20 may be between about 25 mm and about 35 mm in a fully dilated position (e.g., see FIG. 5C). The diameter of the opening 137 formed by the band 20 may be between about 15 mm and about 20 mm in a fully constricted position (e.g., see FIG. 5D).

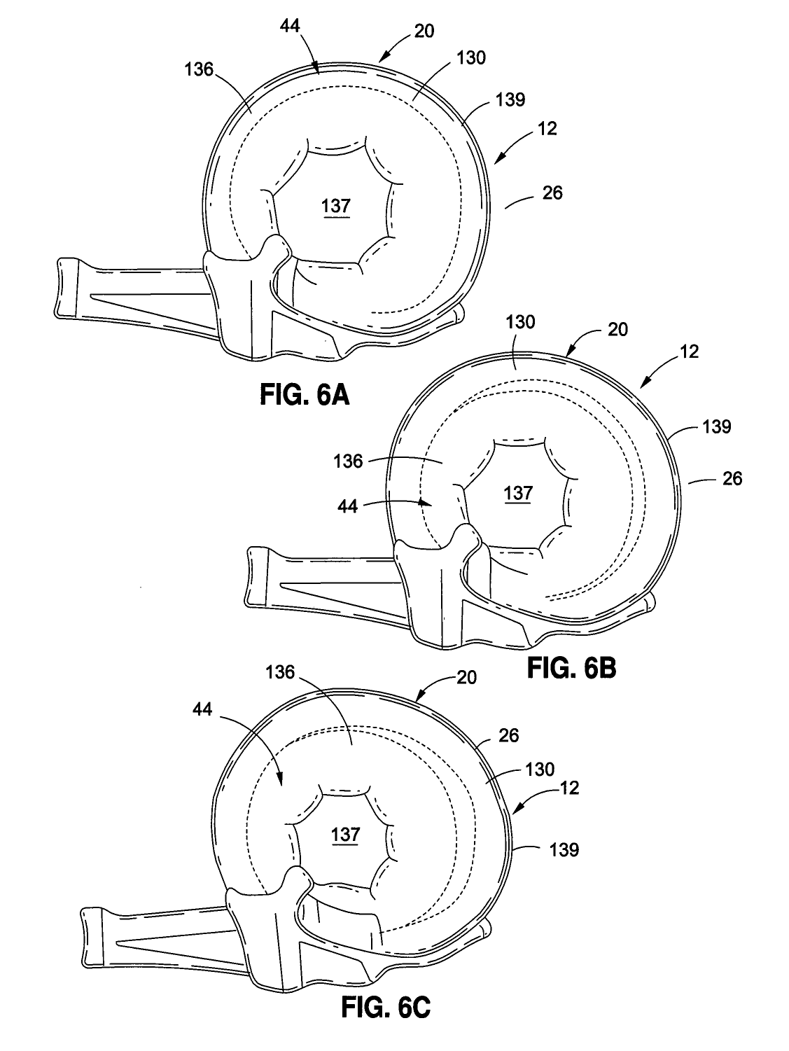

FIGS. 6A, 6B and 6C show the band 20 at progressively increased levels of constriction, with FIG. 6A showing the opening 137 being larger than in FIG. 6B, which shows the opening 137 larger than in FIG. 6C. In the shown embodiments, while the diameter of the opening 137 is adjustable, the diameter of an outer circumferential surface 139 of the band 20 remains relatively fixed during adjustments of the opening 137. The membrane 45 of the contact region 44 stretches or unfolds as described elsewhere herein, as axially compressible material 136 moves apart from the distal element 130 and the band (not visible in FIGS. 6A-6C) and opening 137 constricts (see also FIG. 5D).

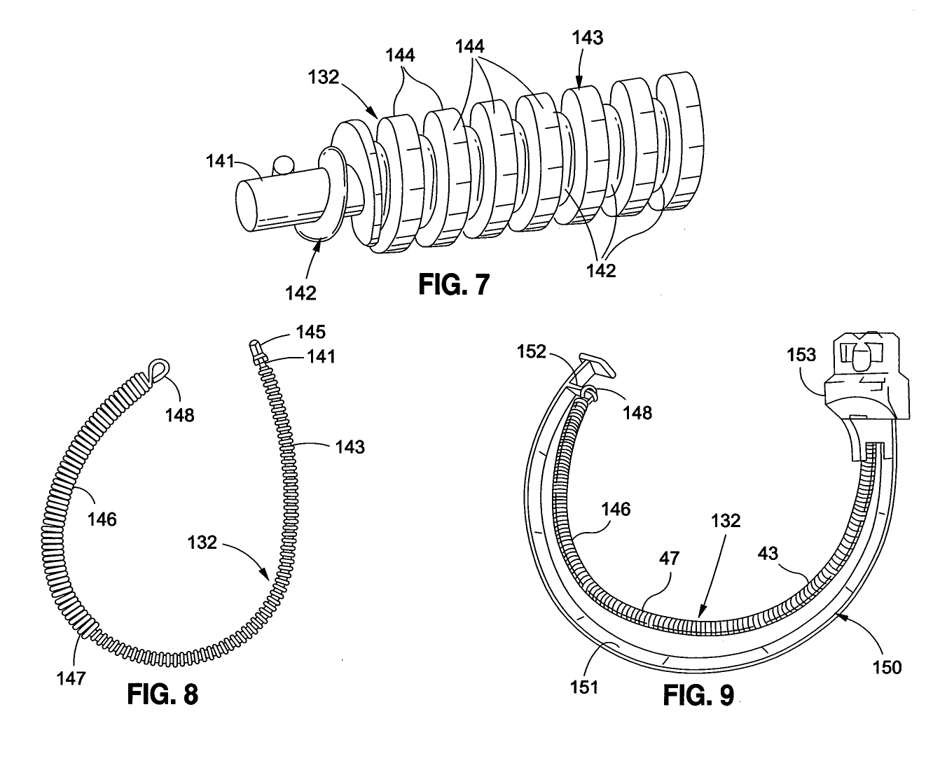

Referring now to FIG. 7, the tension element 132 is described. In some embodiments, the tension element 132 has sufficient flexibility to permit it to be formed into a substantially circular shape, while also being able to transmit the force necessary to adjust the inner diameter of the loop. The tension element 132 may comprise a flexible core 141, for example, comprising a metal alloy wire of circular cross section, on which is fixed, and wound coaxially, at least one un-joined coil spring which defines a screw thread pitch.

The tension element 132 may comprise two un-joined coil springs that form a screw thread: first spring 142, wound helicoidally along the flexible core 141, and second spring 143 of greater exterior diameter. The second spring 143 preferably comprises coils 144 of rectangular transverse section, so as to delineate a flat external generatrix. The first spring 142 is interposed between the coils 144 of the second spring 143 to define and maintain a substantially constant square screw thread pitch, even when the tension element is subjected to bending.

The second spring 143 may be made by laser cutting a cylindrical hollow tube, e.g., made from stainless steel, or alternatively, by winding a wire with a rectangular, trapezoidal or other cross-section. When helically intertwined with the first spring 142, the coils 144 of the second spring 143 are activated with an intrinsic elastic compression force from the adjacent coils of the first spring 142. The first spring 142 is intertwined between the coils of the second spring 143. The first spring 142 is fixedly joined to the flexible core 141 at one end. At the second end, a crimped cap 145 (see FIG. 8) is located a short distance from the ends of the springs 142 and 143 to allow for small extensions, for example, to accommodate flexion of the tension element 132 and/or to limit this extension to keep the thread pitch substantially constant.

Referring now to FIG. 8, a free end of the tension element 132 includes a crimped cap 145. The second spring 143 includes coils having a square transverse section. The flexible core 141 extends through the first and second springs 142 and 143, and terminates close to the crimped cap 145. In one embodiment of the present invention, the tension element 132 further comprises a third spring 146 that is coupled to the flexible core 141, and the first and second springs 142 and 143 at junction 147. The third spring 146 includes a loop 148 at the end opposite to junction 147, which permits the tension element 132 to be fixed at the first end 22 of the band 20 (see also FIG. 5A).

With respect to FIG. 9, the tension element 132 is shown disposed within a skeleton 150 of the band 20. The skeleton 150 includes a layer 151 that forms a distal periphery, an anchor 152 that accepts the loop 148 of the tension element 132, and an actuator housing 153. The skeleton 150 may be made of a high strength moldable plastic.

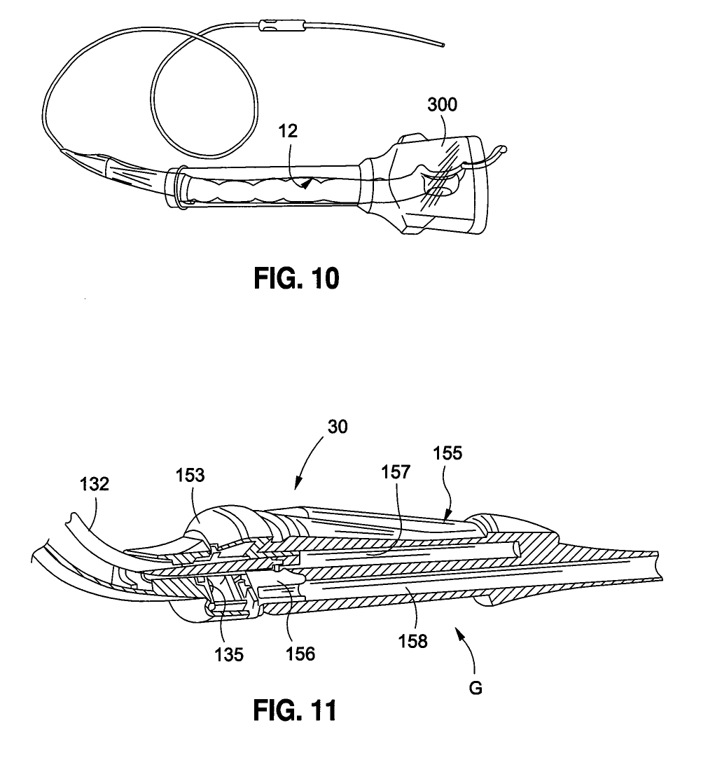

The third spring 146 permits the band 20 to be straightened for insertion through a trocar, for example, an 18 mm trocar, despite a differential elongation of the skeleton 150 and the tension element 132. This feature is illustrated in FIG. 10 which shows the implantable portion 12 (e.g., the band 20) disposed in a trocar 300 in order to facilitate laparoscopic implantation of the band 20.

Referring now to FIG. 11, in the shown embodiment, a connector 30 includes a housing 155 having a recessed portion 156, a tension element cavity 157 and a cable lumen 158. The recessed portion 156 is configured to accept the actuator housing 153 of the skeleton 150, so that as the tension element 132 is drawn through the actuator 135 it extends into a tension element cavity 157. The cable lumen 158 extends through the housing 155 so that the cable may be coupled to the actuator 135. The housing 155 may be grasped in area G using atraumatic laparoscopic graspers during implantation.

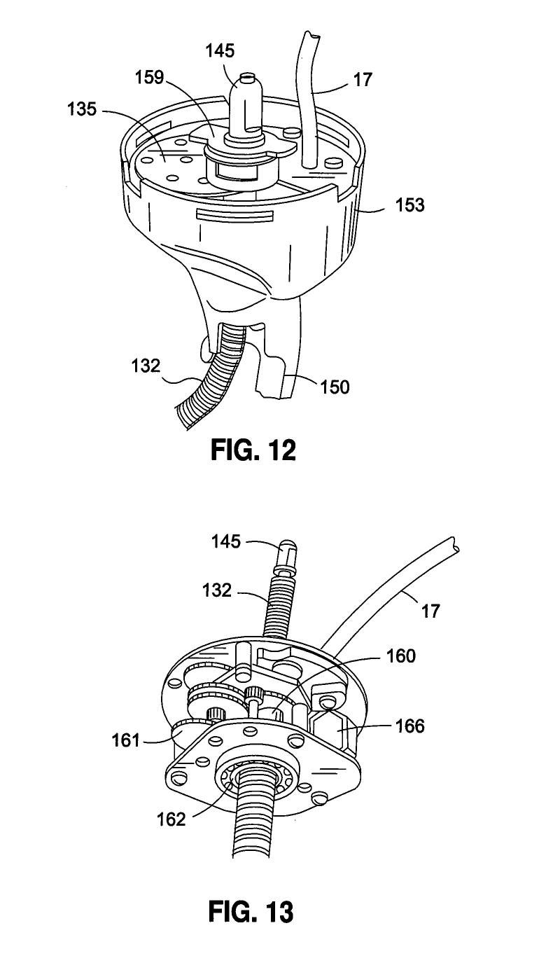

In FIG. 12, the actuator housing 153 of the skeleton 150 is shown with the actuator 135 and the tension element 132 disposed therethrough. The antenna cable 17 is coupled to a motor (not shown) disposed within the actuator housing 153. The tension element 132 is in the fully opened (largest diameter) position, so that the crimped cap 145 contacts a printed circuit board 159 of the reference position switch described below with respect to FIG. 15.

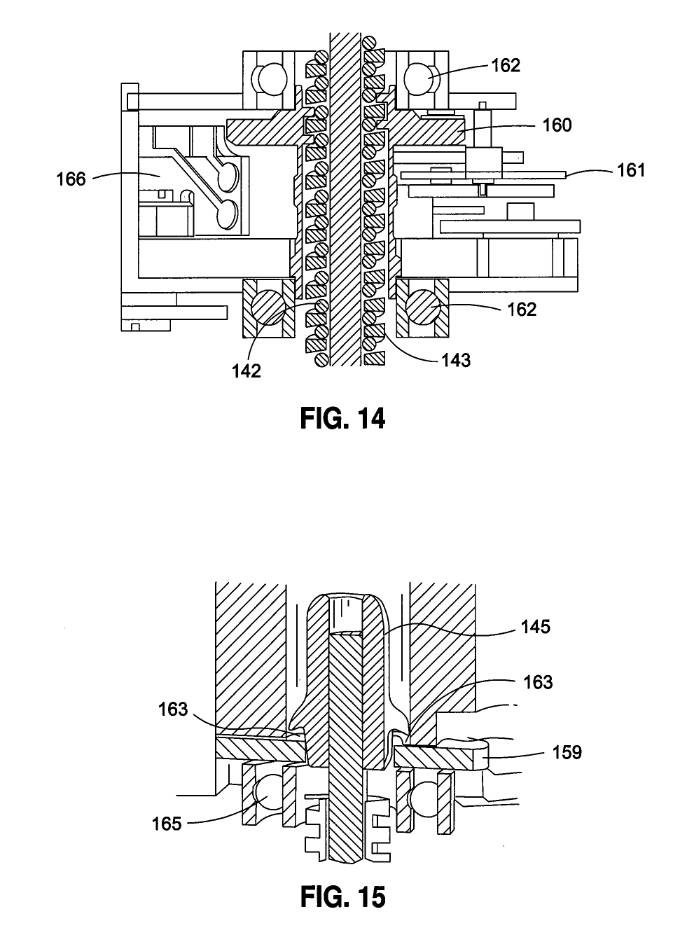

With respect to FIGS. 13 and 14, the actuator 135 includes a motor 166 coupled to the antenna cable 17 that drives a nut 160 through gears 161. The nut 160 is supported by upper and lower bearings 162 to minimize energy losses due to friction. The nut 160 is self-centering, self-guiding and provides high torque-to-axial force transfer. In addition, the nut 160 is self-blocking, meaning that the nut 160 will not rotate due to the application of pushing or pulling forces on the tension element 132. This condition may be achieved by ensuring that the height (h) of the thread divided by the circumference of the screw (27R) is less than the arctangent of the friction coefficient (p):

h/(2πR)<arctan(μ)

The gears 161 preferably are selected to provide good mechanical efficiency, for example, with a reduction factor greater than 1000. In addition, the volume of the actuator depicted in FIGS. 13 and 14 may be quite small, with a total volume less than 1 cm³ and a diameter less than 12.5 mm, so that the device may easily pass through a standard trocar. In a preferred embodiment, the gears 161 are selected to provide a force of more than 2 kg on the screw thread of the tension element 132 at an electrical consumption of only 50 mW. The gears 161 and other components of the actuator 135 may be made of stainless steel or other alloys like Arcap (CuNiZn), or can be gold plated to permit operation in the high humidity likely to be encountered in a human body.

The motor 166 employed in the actuator 135 may comprise a Lavet-type high precision stepper motor with a flat magnetic circuit, such as are used in watches. The motor 166 may be a two phase (two coil) motor that permits bi-directional rotation, has good efficiency, and may be supplied with a square wave signal directly by the microcontroller circuitry within antenna/controller pod 15, thus eliminating the need for an interface circuit. Alternatively, the motor employed in the actuator 135 may be of a brushless DC type motor. In addition, the motor preferably is compatible with magnetic resonance imaging, i.e., remains functional when exposed to strong magnetic fields used in medical imaging equipment.

Referring now to FIG. 15, a reference position switch of an embodiment of the present invention is described. In one embodiment the actuator of the present invention employs the nut 160 driven by a stepper motor. Thus, there is no need for the system to include a position sensor or encoder to determine the length of the tension element 132 drawn through the actuator 135. Instead, the diameter of the opening 137 may be computed as a function of the screw thread pitch and the number of rotations of the nut 160. At least one reference datum point may be provided which may be calculated by using a reference position switch that is activated when the band 20 is moved to its fully open position. The crimped cap 145 on the free end of the tension element 132 may be used to serve this function by contacting the electrical traces 163 on the printed circuit board 159 (and also limits elongation of the screw thread). The circuit board 159 is disposed just above the bearing 165, which forms part of the actuator 135. When the crimped cap 145 contacts the electrical traces 163 it closes a switch that signals the implantable controller that the band 20 is in the fully open position.

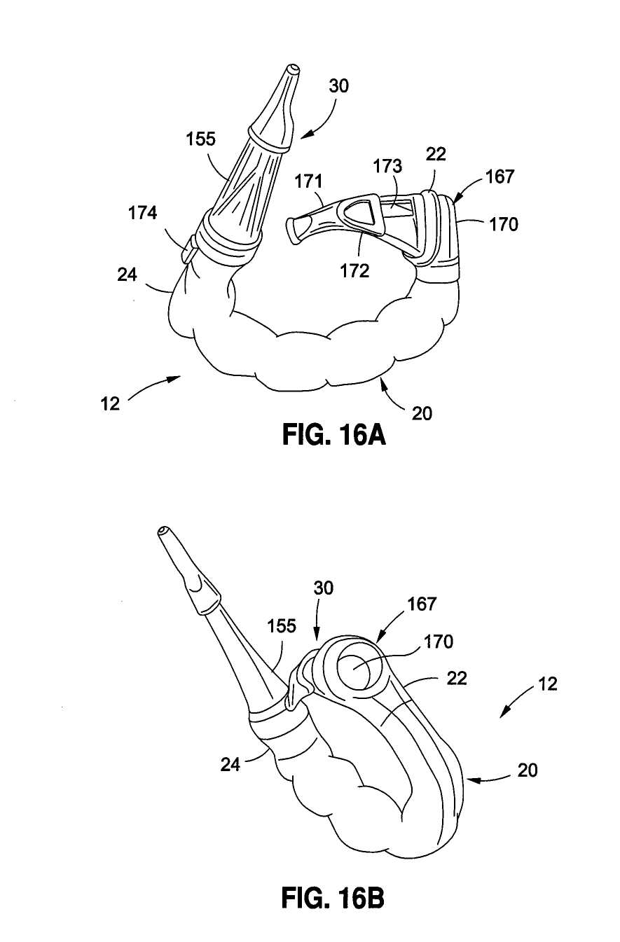

Referring now to FIGS. 16A and 16B, a clip 30 may include a clip element 167 on the first end 22 of the band 20 and the housing 155 on the second end of the band 20. The clip element 167 includes an aperture 170, a tab 171 having a hinge 172 and a slot 173. The aperture 170 is dimensioned to accept the housing 155 on the second end 24 of the band 20, while the slot 173 is dimensioned to accept a flange 174 disposed on the second end 24.

An example of a method of coupling the first end 22 with the second end 24 during implantation of the band 20 is now described. To couple the first end 22 and the second end 24, the clip element 167 is grasped by the tab 171, and the tag 18 of the pod 15 (see FIG. 1) is inserted through the aperture 170. The clip element 167 is then pulled towards the second end 24 so that the housing 155 passes through the aperture 170 while the housing 155 is grasped with atraumatic forceps; the conical shape of the housing 155 facilitates this action. The force is applied to the tab 171 until the slot 173 captures the flange 174, thereby securing the first and second ends 22, 24 in the closed position. The physician may subsequently choose to disengage the slot 173 from the flange 174 by manipulating the tab 171 using laparoscopic forceps, for example, to reposition the band 20. In some embodiments, forces inadvertently applied to the tab 171 in an opposite direction cause the tab 171 to buckle at the hinge 172, but do not cause the flange 174 to exit the slot 173. Accordingly, the hinge 172 of the tab 171 prevents accidental opening of the clip 30 when the tab 171 is subjected to forces that cause the tab 171 to fold backwards away from the housing 155 such as may arise due to movement of the patient, the organ, or bolus of fluid passing through the organ.

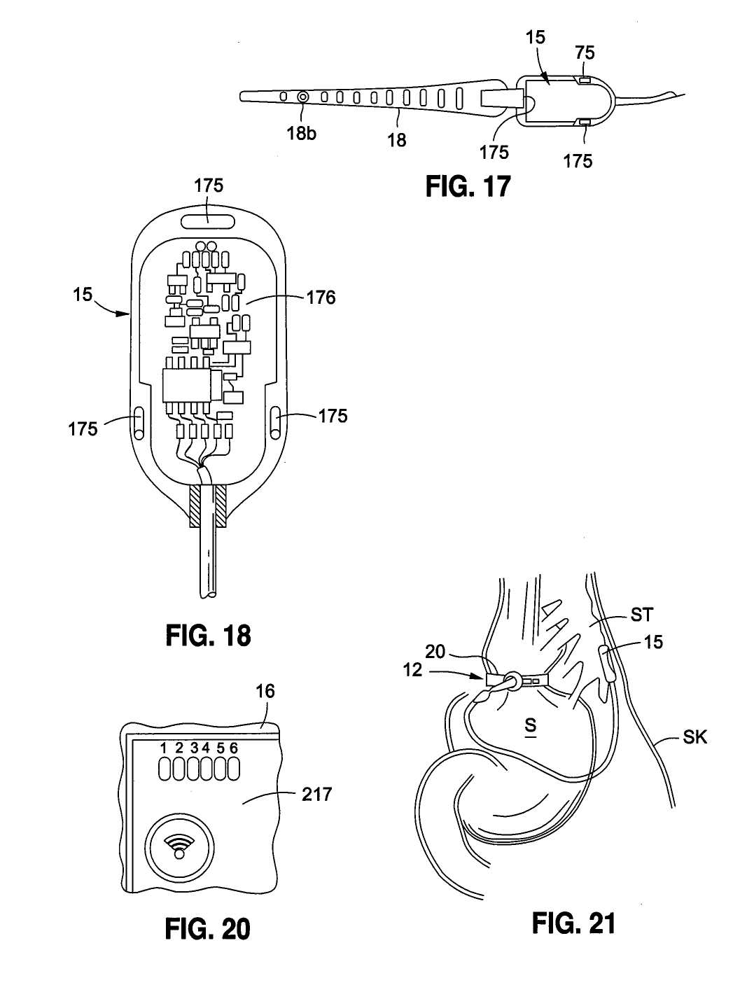

With respect to FIGS. 17 and 18, the removable tag 18 of the antenna/controller pod 15 may include apertures 175. The tag 18 comprises a grip structure that facilitates manipulation and placement of the pod 15 during implantation; after which the tag 18 is removed, for example, using a scissors cut. The tag 18 also includes aperture 18b that allows the use of a suture thread to assist in passing the antenna/controller pod 15 behind the stomach. The holes 175 also are dimensioned to be compatible with standard suture needles from size 1-0 to 7-0 to permit the pod 15 to be sutured to the patient's sternum, thereby ensuring that the pod 15 remains accessible to the external antenna and cannot migrate from a desired implantation site.

As shown in FIG. 18, the antenna/controller pod 15 encloses the printed circuit board 176 that carries the antenna and microcontroller circuitry of the band (not shown). The antenna receives energy and commands from the external control 16 (see FIG. 1), and supplies those signals to the microcontroller, which in turn powers the motor 166 of the actuator 135 (FIGS. 12 and 13). The circuitry of the antenna/controller pod 15 uses the energy received from the incoming signal to power the circuit, interprets the commands received from the external control 16, and supplies appropriate signals to the motor 166 of the actuator 135. The circuit also retrieves information regarding operation of the motor 166 of the actuator 135 and relays that information to the external control 16 via the antenna. The printed circuit board 176 may be covered with a water-resistant polymeric covering, e.g., Parylene, to permit use in the high (up to 100%) humidity environment encountered in the body.

The antenna/controller pod 15 may include a mechanical closure system that is augmented by silicone glue so that the pod 15 is fluid tight. This silicone glue also is used to protect the soldered wires.

The actuator 135 may be linked to subcutaneous antenna/controller pod 15 to receive a radio frequency control and power signal. In one embodiment, the motor 166 of the actuator 135 has no internal energy supply, but rather is powered by the receiving circuit of the antenna through a rechargeable energy storage device, such as a capacitor. For example, the receiving circuit converts radio frequency waves received from external control 16 via the antenna into a motor control and power signal. In another embodiment the actuator 135 may be driven via an implantable rechargeable battery.

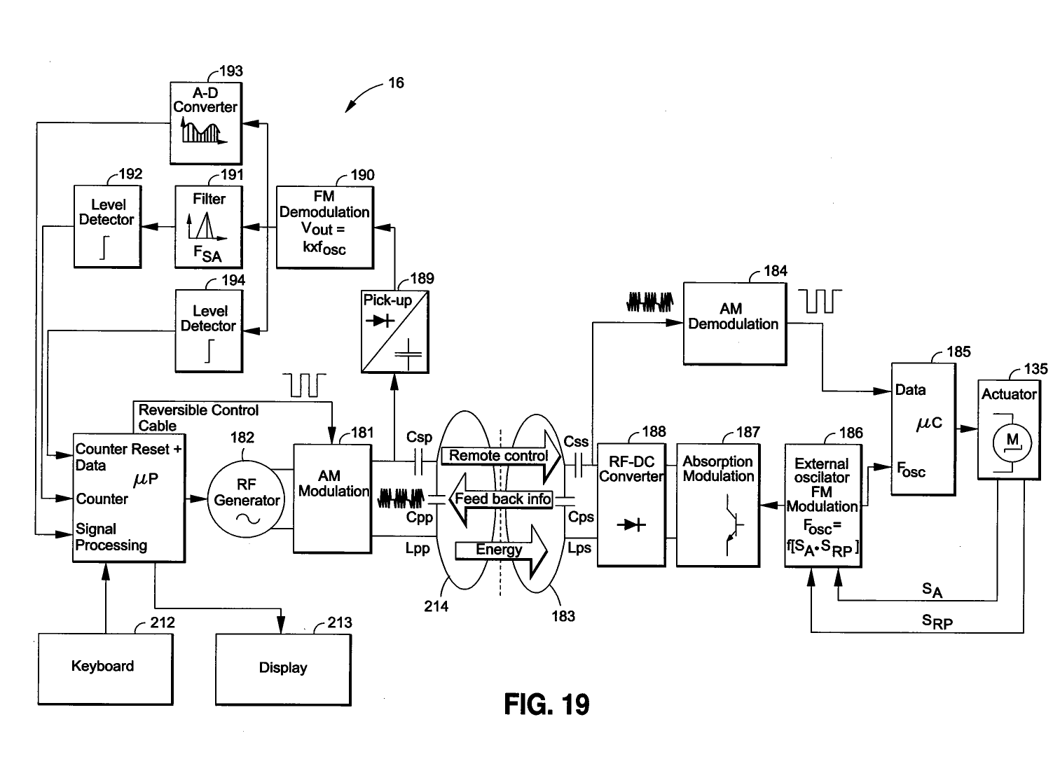

Referring to FIG. 19, one suitable arrangement of circuitry that may be employed in the external control 16 of the present invention is described herein. The external control 16 includes a microprocessor 180 coupled to a keyboard/control panel 212 and a display 213. The external control 16 produces a signal comprising one or more data bytes to be transmitted to the implantable antenna/controller pod (not shown) and the actuator 135.

The external control 16 includes a modulator 181 for amplitude modulation of the RF wave from a RF generator 182, whose signal is emitted by an external antenna 214. The emitted signal or wave is received by the antenna 183 in the antenna/controller pod (not shown), where the AM demodulator 184 extracts the data bytes from the envelope of the received RF signal. The data bytes then are decoded by the microcontroller 185. A special code is used that allows easy decoding of the data by the microcontroller 185, but also provides maximal security against communication failure.

The external oscillator 186, which is a voltage controlled oscillator (VCO), provides a clock signal to the microcontroller 185. The oscillator 186 may comprise, for example, a relaxation oscillator comprising an external resistor-capacitor network connected to a discharging logic circuitry already implemented in the microcontroller or a crystal oscillator comprising a resonant circuit with a crystal, capacitors and logic circuits.

The microcontroller 185 interprets the received instructions and produces an output that drives the motor of the actuator 135. As discussed above, the actuator 135 may comprise a bi-directional stepper motor that drives the nut 160 through a series of reducing gears. In one embodiment, the two coils of the stepper motor of the actuator 135 are directly connected to the microcontroller 185, which receives the working instructions from the demodulator 184, interprets them and provides the voltage sequences to the motor coils. When the supply of voltage pulses to the stepper motor stops, the gears are designed to remain stationary, even if a reverse torque or force is applied to the nut 160 by the tension element 132.

As also described above, use of a stepper motor in the actuator 135 makes it is possible to obtain positional information on the nut 160 and the tension element 132 without the use of sensors or encoders, because the displacement of the tension element 132 is proportional to the number of pulses supplied to the stepper motor coils. Two signals may be employed to ensure precise control, reference position signal S_{RP}, generated by the reference position switch of FIG. 15, and the actuator signal S_{A}.

According to one embodiment, signal S_{A} is the voltage signal taken at one of the outputs of the microcontroller 185 that is connected to the motor coils of the actuator 135. Alternatively, signal S_{A} can be derived from the current applied to a motor coil instead of the voltage, or may be an induced voltage on a secondary coil wrapped around one of the motor coils of the actuator 135. In either case, signal S_{A} may be a pulsating signal that contains information on the number of steps turned by the rotor and further indicates whether blockage of the mechanism has occurred. Specifically, if the rotor of the stepper motor fails to turn, the magnetic circuit is disturbed, and by induction, affects signal S_{A}, e.g., by altering the shape of the signal. This disturbance can be detected in the external control, as described below.

The signals S_{A} and S_{RP} are converted into frequencies using the external oscillator 186, so that the voltage level of signal S_{A} applied to the external oscillator 186 causes the oscillator to vary its frequency F_{OSC} proportionally to the signal S_{A}. Thus, F_{osc} contains all the information of signal S_{A}. When the crimped cap 145 and the tension element 132 are in the reference position (band 20 is fully open), the reference position switch produces reference position signal S_{RP}. The signal S_{RP} is used to induce a constant shift of the frequency F_{OSC}, which shift is easily distinguishable from the variations due to the signal S_{A}.

If the oscillator 186 is a relaxation oscillator, as described above, the signals S_{A} and S_{RP} modify the charging current of the external resistor capacitor network. In this case, the relaxation oscillator may comprise an external resistor-capacitor network connected to a transistor and a logic circuit implemented in the microcontroller 185. With the signals S_{A} and S_{RP}, the goal is to modify the charging current of the capacitor of the RC network to change the frequency of the relaxation oscillator. If the charging current is low, the voltage of the capacitor increases slowly and when the threshold of the transistor is reached, the capacitor discharges through the transistor. The frequency of the charging-discharging sequence depends on the charging current.

If the oscillator 186 is a crystal oscillator, signals S_{A} and S_{RP} modify the capacitor of the resonant circuit. In this case, the crystal oscillator circuit preferably comprises a crystal in parallel with the capacitors, so that the crystal and the capacitors form a resonant circuit which oscillates at a fixed frequency. This frequency can be adjusted by changing the capacitors. If one of these capacitors is a Varicap (a type of diode), it is possible to vary its capacitance value by modifying the reverse voltage applied on it, signals S_{A} and S_{RP} can be used to modify this voltage. In either of the foregoing cases, signals S_{A} and S_{RP} may be used to modify at least one parameter of a resistor-capacitor (RC) network associated with the oscillator 186 or at least one parameter of a crystal oscillator comprising the oscillator 186.

Referring still to FIG. 19, signals S_{A} and S_{RP}, derived from the stepper motor or from the output of the microcontroller 185, may be used directly for frequency modulation by the oscillator 186 without any encoding or intervention by the microcontroller 185. By using the oscillator 186 of the microcontroller 185 as part of the VCO for the feedback signal, no additional components are required, and operation of the microcontroller 185 is not adversely affected by the changes in the oscillator frequency F_{OSC}. The oscillating signal F_{OSC} drives the voltage driven switch 187 for absorption modulation, such that feedback transmission is performed with passive telemetry by FM-AM absorption modulation.

More specifically, the signal F_{OSC} drives switch 187 such that during the ON state of the switch 187 there is an increase in energy absorption by the RF-DC converter 188. Accordingly, therefore the absorption rate is modulated at the frequency F_{OSC} and thus the frequency of the amplitude modulation of the reflected signal or wave detected by the external control 16 contains the information for signal S_{A}. As discussed below, the pickup 189 in the external control 16 separates the reflected signal or wave where it can be decoded by FM demodulation in the demodulator 190 to obtain signal S_{A}′. This method therefore allows the transmission of different signals carried at different frequencies, and has the advantage that the ON state of the switch 187 can be very short and the absorption very strong without inducing an increase in average consumption. In this way, feedback transmission is less sensitive to variation in the quality of coupling between the antennas 183 and 214.

In the external control 16, the feedback signal F_{OSC} is detected by the pickup 189 and fed to the FM demodulator 190, which produces a voltage output V_{OUT} that is proportional to F_{OSC}. V_{OUT} is fed to the filter 191 and the level detector 192 to obtain the information corresponding to the actuator signal S_{A}, which in turn corresponds to the pulses applied to the stepper motor coil. The microprocessor 180 counts these pulses to calculate the corresponding displacement of the tension element 32, which is proportional to the number of pulses.

The signal V_{OUT} also is passed through the analog-to-digital converter 193 and the digital output is fed to the microprocessor 180, where signal processing is performed to detect perturbations of the shape of the feedback signal that would indicate a blockage of the rotor of the stepper motor. The microprocessor 180 stops counting any detected motor pulses when it detects that the actuator is blocked, and outputs an indication of this status. The level detector 194 produces an output when it detects that the demodulated signal V_{ow} indicates the presence of the reference position signal S_{RP} due to activation of the reference position switch. This output induces a reset of the position of the tension element calculated by the microprocessor 180 in the external control. In this way, a small imprecision, e.g. an offset, can be corrected.

As described above, the external control 16 may be configured to transmit both energy and commands to the implantable controller circuitry in the antenna/controller pod 15. The external control 16 may also receive feedback information from the implantable controller that can be correlated to the position of the tension element 132 and the diameter of the loop. The external control 16 and the implantable controller may be configured in a master-slave arrangement, in which the implantable controller is completely passive, awaiting both instructions and power from the external control 16.

Power may be delivered to the implantable pod 15 via magnetic induction. The quality of the coupling may be evaluated by analyzing the level of the feedback signal received by the external control 16, and a metric corresponding to this parameter may be displayed on the signal strength indicator 217 on the control 16, which in the shown embodiment, includes 6 LEDs (corresponding to six levels of coupling). If the coupling between the antennae is insufficient, the motor of the actuator may not work properly.

Referring now to FIG. 21, the band 20 of the presently described system is shown implanted in a patient. The band 20 is disposed encircling the upper portion of the patient's stomach S while the antenna/controller pod 15 is disposed adjacent to the patient's sternum ST. The pod 15 is located in this position beneath the patient's skin SK so that it is easily accessible in the patient's chest area to facilitate coupling of the implanted pod 15 to an external antenna of the control 16.

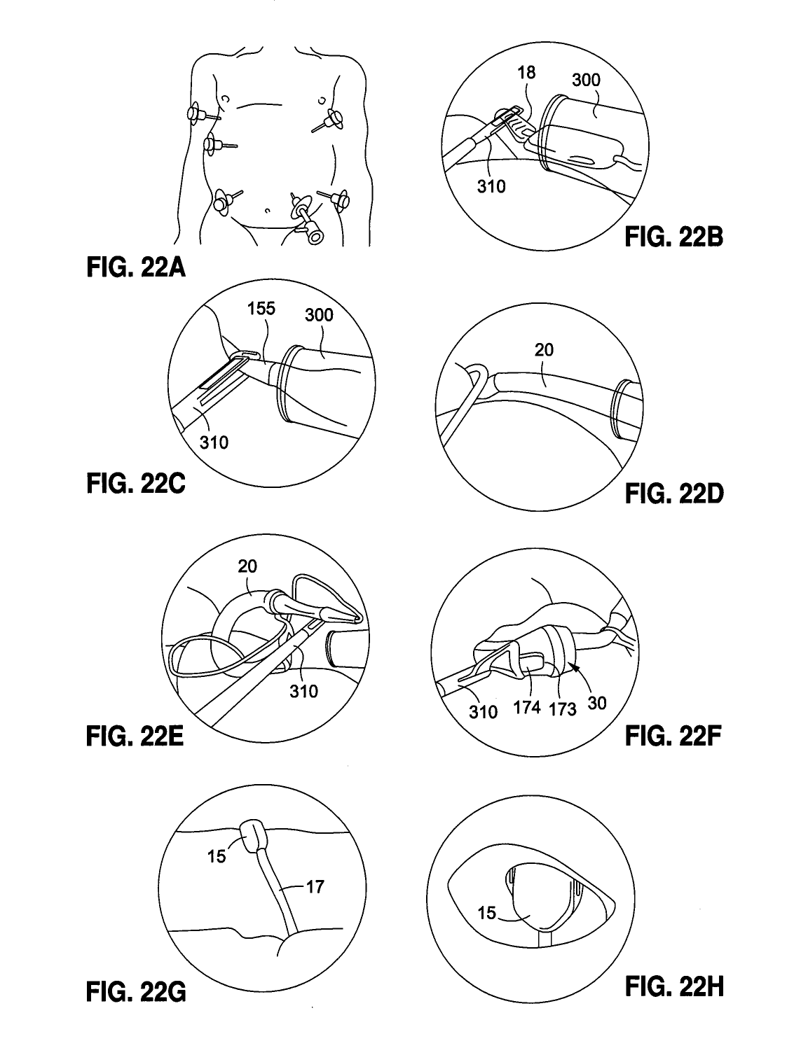

Referring to FIGS. 22A to 22H, a method of implanting the band and the pod of the system of the present invention is described. The method is similar to laparoscopic procedures used to implant previously-known hydraulically-actuated gastric bands.

Access to the abdomen is obtained by using 4 to 6 small holes, generally 10 to 18 mm in diameter, with a trocar inserted in each hole, as depicted in FIG. 22A. A camera and laparoscopic surgical tools are introduced and manipulated through the trocars. In addition, to permit free motion of the surgical tools and camera, the abdomen is inflated with CO₂ to an overpressure of approximately 0.15 bars.

In FIGS. 22B-22E, the band 20 of the implantable portion 12 is straightened (as depicted in FIG. 10) and inserted, antenna first, into the abdomen through an 18 mm trocar. Alternatively, a laparoscopic cannula may be used to make an incision and then withdrawn, and the device is inserted through the opening so created (other instruments also may be used to form this laparotomy). In FIG. 22B, the tag 18 of the antenna/controller pod 15 is shown entering the abdomen through the trocar 300 using atraumatic graspers 310. In FIG. 22C, the housing 155 is shown being drawn into the abdomen through trocar 300, again using atraumatic graspers 310. FIG. 22D shows the band 20 entering the abdomen in an extended position. In FIG. 22E, the band 20 is permitted to resume its arcuate shape.

The band 20 then is manipulated using atraumatic graspers 310 as described elsewhere herein, to secure the band 20 around the upper portion of the patient's stomach until the slot 173 of the clip 30 is engaged with the flange 174, as shown in FIG. 22F. A fold of stomach tissue then may be sutured around the band 20 to prevent migration of the band 20.

Finally, as shown in FIG. 22G, a channel may be formed through the abdominal wall and the antenna/controller pod 15 passed through the channel. The tag 18 then is cut off of the antenna/controller pod 15, and the pod 15 is sutured into position above the patient's sternum, as depicted in FIG. 22H. The trocars then are removed, and the band 20 may be activated to adjust the diameter of the inner diameter as desired by the physician.

The process of removing the band 20 involves substantially reversing the sequence of steps described above, and may be accomplished non-destructively. In particular, a plurality of cannulae into the abdominal cavity and the abdominal cavity then insufflated to create a pneumoperitoneum. Using laparoscopic graspers, the clip 30 may be unclipped and the band 20 removed from a position encircling the patient's stomach. The band 20 may then be straightened and withdrawn from the abdominal cavity either through one of the plurality of cannulae or via a laparotomy.

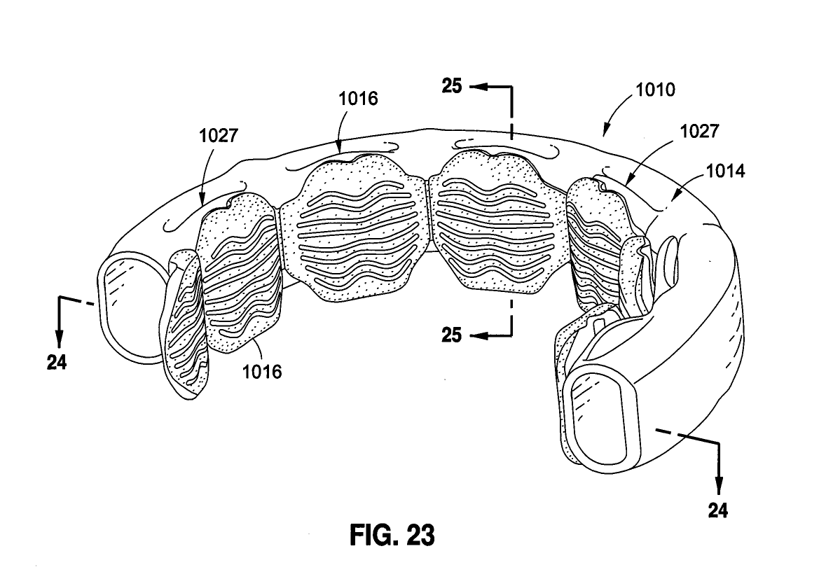

FIGS. 23 through 25 illustrate an alternative contact region 1010 of a gastric banding system of the present invention. The contact region 1010 may be identical to the contact region 44 except as explicitly described below. The contact region 1010 can replace the contact region 44 described and shown, for example, in FIGS. 3 and 3A, in the system 10.

The contact region 1010 comprises a membrane 1014 which may be substantially identical to the membrane 45 described and shown elsewhere herein. In this embodiment however, cushions 1016, which may be made of the same incompressible materials as cushions 60, are affixed to an external surface of the membrane 1014 and define at least a portion of the stomach-facing surface of the contact region 1010. The cushions 1016 may be individually molded to, or molded as a whole, directly to the membrane 1014 using conventional molding techniques, for example, conventional overmolding techniques.

In a specific embodiment, the cushions 1016 are made of silicone elastomer having a hardness of about 10 Shore A and the membrane 1014 is made of silicone elastomer having a hardness of about 30 Shore A.

Alternatively, the membrane 1014 may be made of a silicone elastomer of a different hardness, such as, for example, about 20 Shore A to about 45 Shore A. Alternatively still, the cushions can be made of an even softer silicone elastomer, such as about 5 Shore A or about 1 Shore A. Alternatively, the cushions or the membrane can be made of other suitable implantable materials.

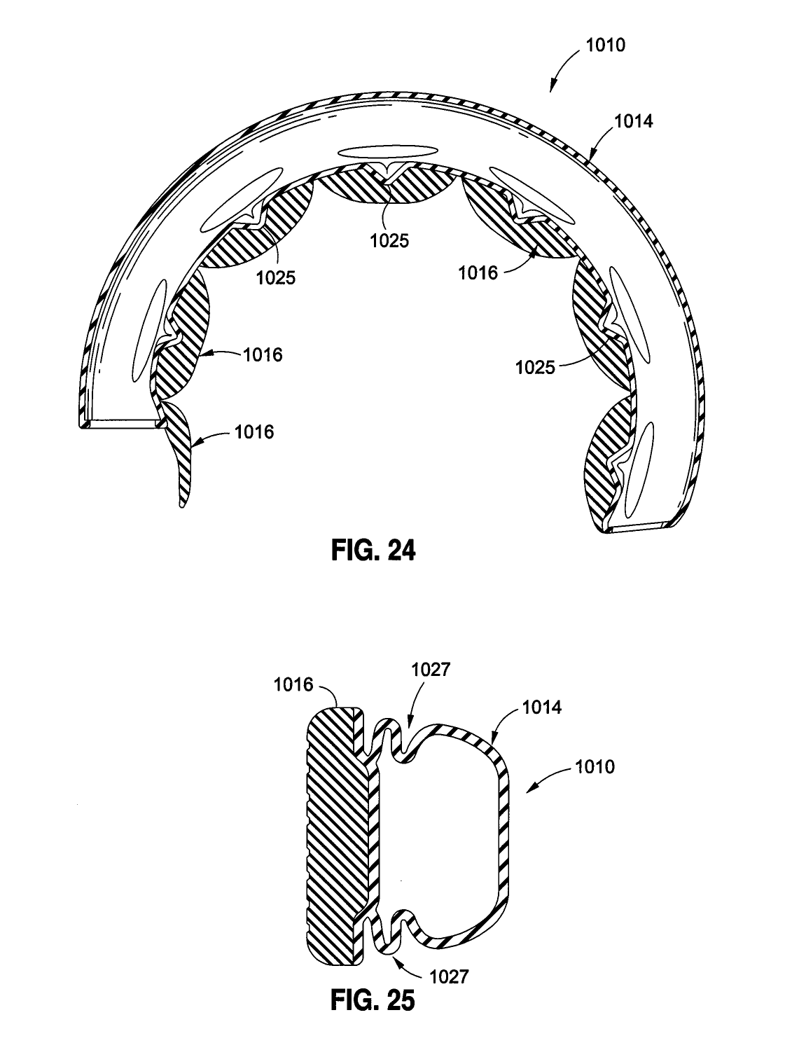

FIGS. 24 and 25 are cross-sectional views of the contact region shown in FIG. 23 taken along line 24-24 and line 25-25, respectively.

Another feature of this embodiment of the invention is shown in FIG. 24. Specifically, the membrane 1014 may includes a structural support, for example, a wedge 1025 located at the interface between the membrane 1014 and each of the cushions 1016. The wedges 1025 may provide an increased surface area on which the cushions are molded thereby providing additional adherence and/or support between the membrane 1014 and the cushions 1016. Like membrane 45, the membrane 1014 includes corrugations 1027 for facilitating unfolding or expansion of the membrane 1014 during adjustment of the band 20.

Another advantageous feature of this embodiment is shown in FIGS. 26-27A. In some embodiments, the cushions 60 and the tension segments 52 form an inner circumference of the loop configuration having a generally star-shape, defined by the contact region, as shown in FIG. 26. The stomach lumen is indicated by numeral 1033. During constriction of the band, which is shown dilated in FIGS. 26 and 26A and constricted in FIGS. 27 and 27A, the adjacent incompressible cushions 60 form a progressively narrowing angle, for example, a progressively narrowing substantially V-shaped surface having convex, arcuate surfaces defined by the cushions 60. The tension segments 52 are located between the adjacent cushions 60 and form the vertices of the angles.

While not wishing to be bound by any particular theory of operation, it is believed that the structure of the contact member 44 and at least partially due to the incompressibility of the cushions 60 enables the band 20 to constrict about the stomach without pinching the tissue. For example, as shown in FIGS. 27 and 27A, the stomach tissue does not become entrapped between adjacent cushions 60. During constriction of the band 20, the convex stomach-facing surfaces maintain their shape and form no gaps, while folding inwardly toward one another. This mechanism and structure causes the tissues of the stomach constricted without the tissues becoming entrapped and/or pinched. This progressive V-shape acts differently from mechanical pliers.

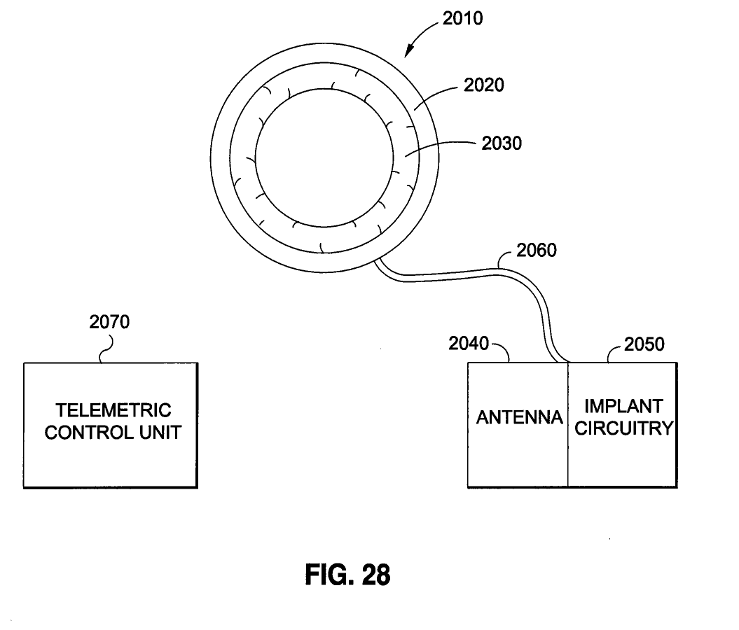

FIGS. 28 through 32 illustrate yet another alternative contact region of a gastric banding system of the present invention, such as an inflatable compartment 2030. The inflatable compartment 2030 may be identical to the contact region 44 except as explicitly described below. The inflatable compartment 2030 can replace the contact region 44 described and shown, for example, in FIGS. 3 and 3A, in the system 10. In exemplary embodiments, the inflatable compartment 2030 may be used in connection with the exemplary inner mechanism shown in FIGS. 5A-5D which enables adjustment of the inner circumference of the loop configuration.

Preliminarily, and as already discussed herein, obesity is a matter of worldwide concern. Various approaches have been taken to address the underlying causes of obesity, including adjustable gastric banding. There are at least three types of adjustable gastric bands, namely, remotely adjustable bands (RABs), hydraulic adjustable bands (HABs), and hydraulic remotely adjustable bands (HRABs), each having its own respective advantages and disadvantages.

An exemplary RAB, such as described herein, comprises a stepper motor which rotates around a flexible screw within the ring of the band to adjust the band through a variety of diameters. As the stepper motor drives forward it reduces the stoma of the band and when it reverses it increases the stoma of the band. The adjustment is telemetrically controlled by an external controller. The band has an EPTFE cushion which is covered by a silicone sheath to contact the stomach. While RABs are relatively low profile, the stomach interface is not as soft as with HABs and HRABs, there is a greater potential for slippage than with HABs and HRABs, and there is no override mechanism in the event of an emergency as with HABs and HRABs.

An exemplary HAB comprises saline solution inside of one or more inflatable silicone shells positioned on the stomach surface of the ring of the band to adjust the band through a variety of diameters. As the shell is inflated it reduces the stoma of the band and when it is deflated it increases the stoma of the band. Saline solution is added or removed from the shell via an access port fixed beneath the skin of the patient in the abdomen on the rectus muscle sheath using a fine needle to find the right level of restriction. While HABs are characterized by a soft saline solution stomach interface, with which physicians are very comfortable, and may incorporate an override mechanism in the event of an emergency, HABs are bulkier than RABs and the access port bump is often unattractive, especially after patients lose weight.

An exemplary HRAB also comprises saline solution inside of one or more inflatable silicone shells positioned on the stomach surface of the ring of the band to adjust the band through a variety of diameters. However, rather than via an access port fixed beneath the skin of the patient's abdomen, saline solution is added or removed from the shell via a pump, active valves, electronics, and reservoir. Similar to HABs, while HRABs are characterized by a soft stomach interface and may incorporate an override mechanism in the event of an emergency, the components of HRABs require a larger overall implant size than both HABs and RABs, which is viewed as a disadvantage.

An object of the present invention is therefore to combine the best features of RABs, HABs, and HRABs in a novel and non-obvious manner to thereby reduce the disadvantages of each design.

With reference to FIG. 28, an implantable banding system in accordance with exemplary embodiments of the present invention comprises a gastric band 2010 and: (i) a mechanical adjustment mechanism 2020, (ii) one or more inflatable compartments 2030, (iii) an antenna 2040 and implant circuitry 2050, (iv) a connector 2060, and (v) a telemetric control unit 2070. The inflatable compartments 2030 can include, for example, a fixed volume of fluid. Not shown in FIG. 28, an implantable banding system in accordance with exemplary embodiments of the present invention may optionally further comprise, inter alia, one or more of: (i) an override mechanism, (ii) a pressure monitoring mechanism, (iii) a self-adjusting mechanism, and (iv) a drug delivery mechanism.

An implantable banding system in accordance with exemplary embodiments of the present invention comprises a mechanical adjustment mechanism 2020, the same as or similar to the exemplary inner mechanism shown in FIGS. 5A-5D which enables adjustment of the inner circumference of the loop configuration. Exemplary mechanical adjustment mechanisms are configured to adjust the gastric band 2010 through a variety of diameters. In accordance with exemplary embodiments, the mechanical adjustment mechanism 2020 comprises an outer portion of the ring of the gastric band 2010 or implantable banding system. An exemplary mechanical adjustment mechanism 2020 comprises a stepper motor which rotates around a flexible screw within the ring of the band to adjust the gastric band 2010 through a variety of diameters. As the stepper motor drives forward it reduces the stoma of the band and when it reverses it increases the stoma of the band. The adjustment is telemetrically controlled by a telemetric control unit. An exemplary mechanical adjustment mechanism 2020 is described in U.S. Publication No. 2005/0143766, to Bachmann et al., which is hereby incorporated by reference for all purposes in its entirety. In general, a mechanical adjustment mechanism 2020 is any device designed or otherwise configured to controllably restrict the band circumferentially about the stomach.

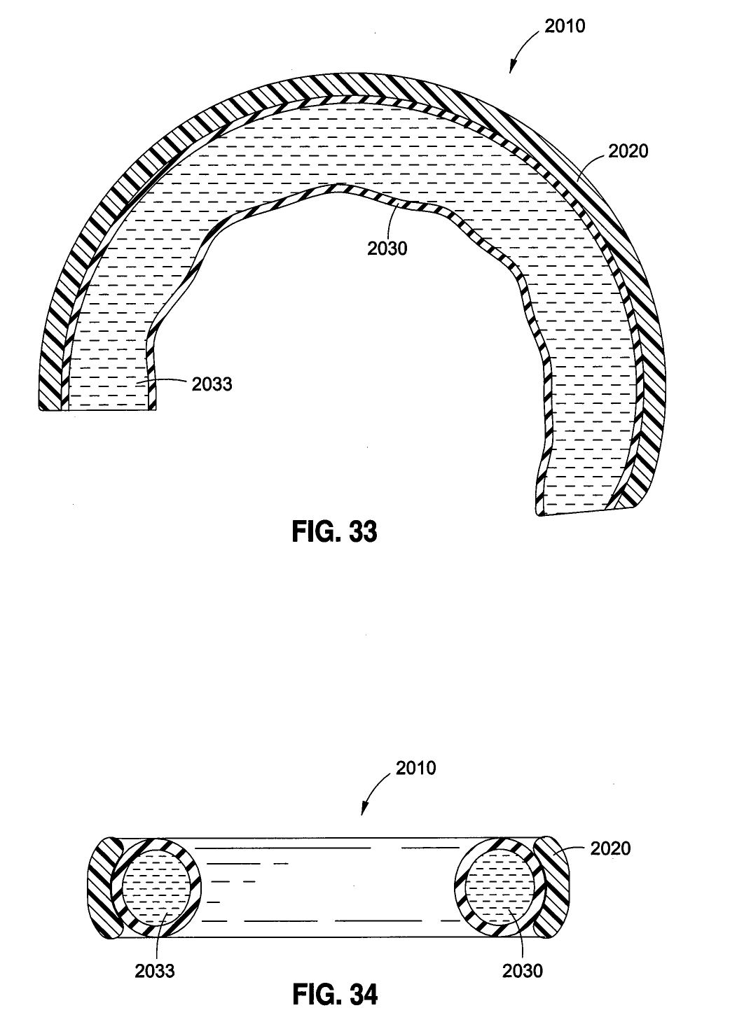

An implantable banding system in accordance with exemplary embodiments of the present invention comprises one or more inflatable compartments 2030. Exemplary inflatable compartments 2030 are positioned on the stomach surface of the ring of the band and configured to provide a soft stomach interface. An exemplary inflatable compartment 2030 may comprise a silicone shell or balloon filled completely or partially with a fluid, e.g., saline solution, water or the like. The fluid can be, for example, a fixed volume of saline or a gel. The saline can be noncompressible, but soft. In accordance with exemplary embodiments, the fluid compartment comprises an inner portion of the ring of the gastric band 2010 or implantable banding system. In accordance with exemplary embodiments, the inner fluid compartment of the ring of the gastric banding element is bonded to, or overmolded onto, the outer mechanical adjustment mechanism either directly or with a silicone sheath intermediary element. As can be seen in the sectional views of the gastric band 2010 in FIGS. 33 and 34, the one or more inflatable compartments 2030 can be filled with fluid 2033.

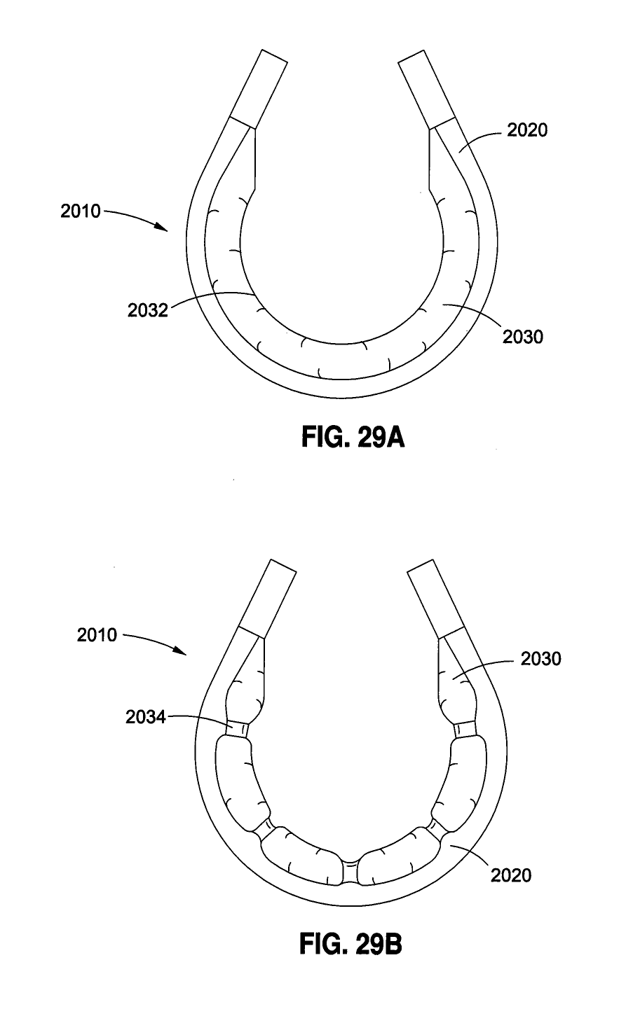

With momentary reference to FIGS. 29A and 29B, in various embodiments, an inflatable compartment 2030 may comprise one or more smooth features 2032 or fatigue-resistant features 2034, such as those described in U.S. Publication No. 2005/0082793, to the present inventor Birk, and U.S. Publication No. 2005/0192531, to the present inventor Birk, both of which are hereby incorporated by reference for all purposes in their entireties. In general, an inflatable compartment 2030 is any device designed or otherwise configured to be a cushioning element between the mechanical adjustment mechanism and the stomach.

An implantable banding system in accordance with exemplary embodiments of the present invention comprises an antenna 2040 configured to telemetrically communicate with the telemetric control unit, and implant circuitry 2050 configured to process information received by the antenna 2040 and thereby control the mechanical adjustment mechanism. In accordance with exemplary embodiments, the antenna 2040 is a low profile antenna or the like, and is attached to the tissue on top of the sternum. Even in morbidly obese patients, the tissue on top of the sternum is a consistent thickness and does not usually exceed several centimeters. By placing the antenna 2040 in this location, the distance between the implant and the telemetric control unit's antenna 2040 is short and a predictable distance. The shallow depth substantially reduces the power requirements necessary to power the band and allows for a smaller external inductive antenna 2040 to power the implant.

An implantable banding system in accordance with exemplary embodiments of the present invention comprises a connector 2060 through which the antenna 2040 and the implant circuitry 2050 are in communication with the mechanical adjustment mechanism 2020.

In exemplary embodiments, the implantable banding system is telemetrically powered, e.g., by RF. In other embodiments, the implantable banding system is powered by one or more of a battery, rechargeable or otherwise, a capacitor, and a fuel cell. In accordance with various aspects of an exemplary embodiment, the power source is recharged by one or more of motion, a chemical change, and a temperature change. For example, in exemplary embodiments, the implantable banding system is powered by one or more of the following: (i) kinetic energy created by body motion stored onto a capacitor, (ii) an implanted fuel cell, (iii) an implanted power source powered by chemistry of the body, (iv) an implanted power source powered by temperature change, and (v) implanted batteries that can be recharged by direct contact.

An implantable banding system in accordance with exemplary embodiments of the present invention comprises a telemetric control unit 2070. In exemplary embodiments, the telemetric control unit 2070 is configured to communicate remotely with the implant circuitry 2050 via the antenna 2040. In exemplary embodiments, the telemetric control unit 2070 provides for wireless control of one or more of mechanical adjustment mechanism 2020, the override mechanism, the pressure monitoring mechanism, the self-adjusting mechanism, and the drug delivery mechanism.

In exemplary embodiments, the telemetric control unit 2070 is powered by alternating current, direct current, e.g., one or more of a battery, rechargeable or otherwise, a capacitor, and a fuel cell.

In one embodiment, the gastric band 2010 can be, for example, a RAB with a fluid in the cushions. For example, the gastric band 2010 can be a RAB with inflatable compartments 2030 including fluids.

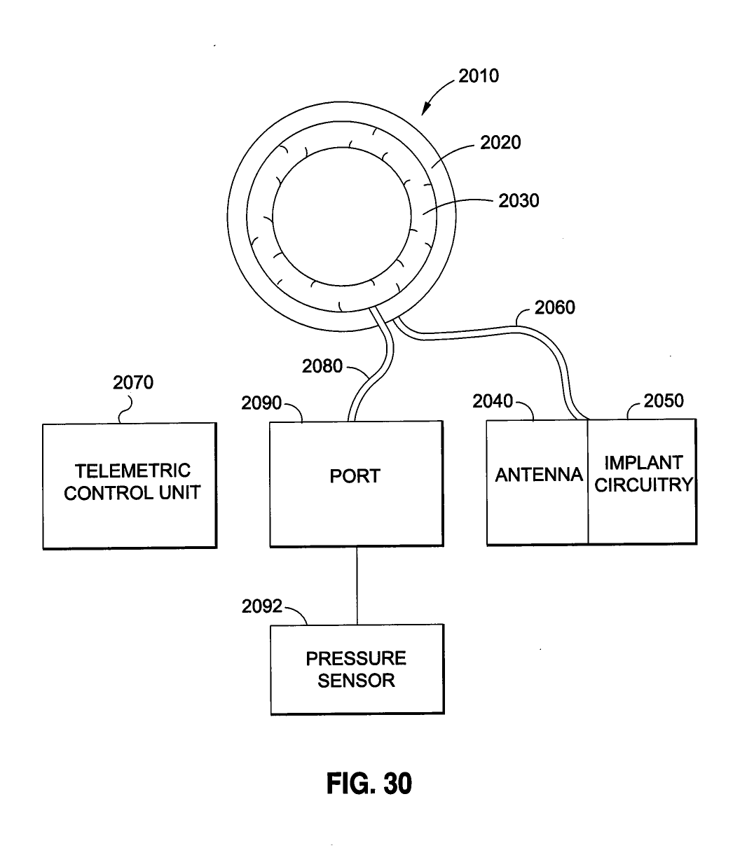

Turning now to FIG. 30, an implantable banding system in accordance with exemplary embodiments of the present invention optionally comprises an override mechanism. In exemplary embodiments, an override mechanism is configured to open the band in the event that there is an electrical failure, a mechanical failure, a power failure, a software failure, a hardware failure, or in the event that a telemetric control unit is not available. In an exemplary override mechanism, the fixed-volume compartment is attached to a port 2090 via tubing 2080 which is implanted in the body to allow for easy access to rapidly adjust the band open in the case of an emergency. The port 2090 can be, for example, a small low profile port. The port 2090 can be placed along side antenna 2040 for easy access.

An implantable banding system in accordance with exemplary embodiments of the present invention optionally comprises a pressure sensor 2092. The pressure sensor 2092 can be connected to, or located within the port 2090. Since the port 2090 can be in fluid communication with the gastric band 2010, the pressure sensor 2092 can detect internal band pressure of the gastric band 2010 and can collect, for example pressure data. In addition, the pressure sensor 2902 can be located, for example, within the gastric band 2010, such as on or within the inflatable compartment 2030, or at an exterior of the inflatable compartment 2030.

In exemplary embodiments, the implantable banding system comprises additional sensors in addition or instead of the pressure sensor 2092 positioned within the inner fluid compartment(s) configured to monitor a parameter of the fixed volume of fluid, optionally generate an adjustment signal based on the parameter and one or more parameter control limits, and optionally either automatically activate the mechanical adjustment mechanism based on the adjustment signal or transmit the adjustment signal to the telemetric control unit. In exemplary embodiments, the parameter may be selected from a pressure, a fill volume, a stress, a strain, a linear measurement, and/or combinations thereof. The data from the pressure sensor and/or other sensors can be transmitted, for example, to the implant circuitry 2050 and/or the telemetric control unit 2070.

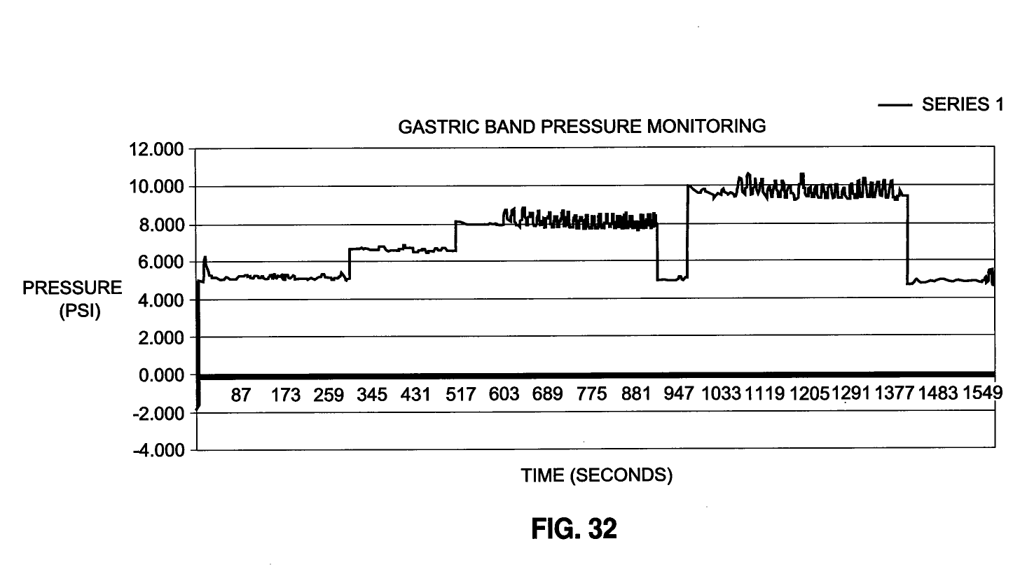

For example, the pressure sensor 2092 can collect pressure data as seen in FIG. 32. In FIG. 32, a needle can be placed in the port 2090 and connected to the pressure sensor 2092. The pressure data can indicate when a patient was at a constriction that was inducing satiety, or when the gastric band 2010 was only slightly over-constricted. This can be seen by the very little variation in pressure between the time period 1 second and 517 seconds. The variation of the pressure can be, for example, less than 0.1 psi. When the gastric band was adjusted to the point where the gastric band was too constricted and the patient felt slightly uncomfortable, there was a greater variation in the pressure data as can be seen in the time period between 603 seconds and 861 seconds.

When the patient swallows water, gradual pressure curves could be seen. As the gastric band 2010 was increasingly constricted, the intra-band pressure response increased in variation as can be seen in the time period between 1033 seconds and 1377 seconds. In some situations, the pressure can vary, for example, by as much as 2 psi. This can provide, for example, the caregiver a new diagnostic tool to determine the optimum level of adjustment based on the pressure response in the gastric band 2010.

Thus, the implant circuitry 2050 can analyze the pressure data from the pressure sensor 2092, to determine whether to constrict or relax the gastric band 2010. In one embodiment, the implant circuitry 2050 can incrementally constrict the gastric band 2010 a nominal constriction and analyze the pressure data for variation information such as standard deviation or greatest difference between maximum and minimum values. If the implant circuitry 2050 determines that the variation information such as the standard deviation is too great, then the implant circuitry 2050 can relax the gastric band by an incremental amount. Any adjustment to the gastric band 2010 can be stored as adjustment data by the implant circuitry 2050. The adjustment data and/or the pressure data can be accessed, for example, by the telemetric control unit 2070 or other external device. The telemetric control unit 2070 can pass along the pressure data and/or the adjustment data to an external device, such as a caregiver's computer. The telemetric control unit 2070 and/or the caregiver's computer can see, for example, a graph such as that depicted in FIG. 32 indicating the pressure data. Thus, the gastric band 2010 can be fully self-automated and self-adjusting.

In one embodiment, the pressure data can be, for example, real-time data. In addition, the pressure data can correspond, for example, to the patient swallowing water and can indicate peristalsis. This can allow the caregiver to visualize the pressure curves generated inside the gastric band 2010 during the adjustment. In addition, average pressure, pressure standard deviation, pressure minimum and maximum measured over time can be collected and transferred to the telemetric control unit 2070 for display.

In one embodiment, the implant circuitry 2050 can be triggered by the telemetric control unit 2070 located at the caregiver's office. Once triggered, the implant circuitry 2050 will proceed to adjust the gastric band 2010 and allow, for example, the caregiver to observe the adjustment results.

Furthermore, the implant circuitry 2050 can also allow for an automatic adjustment of the gastric band 2010 when there is an obstruction. For example, the caregiver or the patient can trigger the implant circuitry 2050 using the telemetric control unit 2070. The implant circuitry 2050 can detect an abnormally high pressure in the gastric band 2010 and to relax the gastric band 2010 to relieve pressure on the patient's stomach. After the obstruction has passed, the implant circuitry 2050 can be triggered again. The constriction of the gastric band 2050 can then be appropriately increased. If there is an ideal pressure for the gastric band 2010 for the specific patient, then the implant circuitry 2050 can also periodically monitor the pressure data to ensure that the gastric band 2010 is operating at the appropriate pressure.

In addition, the caregiver can also adjust the operating parameters of the gastric band 2010 and/or the implant circuitry 2050. For example, the caregiver can increase or decrease the pressure threshold of the gastric band 2010. In addition, the caregiver can increase or decrease the thresholds for the other data detected by the other sensors in the gastric band 2010. For example, the implant circuitry 2050 may be monitoring between 2 psi and 3 psi when the caregiver queries or triggers the implant circuitry 2050 using, for example, the telemetric control unit 2070. The caregiver can adjust the implant circuitry 2050 to monitor between 4.5 and 5.5 psi instead using the telemetric control unit 2070. The implant circuitry 2050 can then increase or decrease the pressure of the gastric band 2010 accordingly so that it is within 4.5 and 5.5 psi. The implant circuitry 2050 can, for example, increase or decrease the amount of constriction by the gastric band 2010.

For example, the implant circuitry will draw power from an implanted battery to allow for the adjustment and will also activate, for example, the check valves in the gastric band 2010 to open the check valves. To constrict the gastric band, the implant circuitry 2050 can instruct the stepper motor to constrict the gastric band 2010. To relax the gastric band 2010, the implant circuitry 2050 can instruct the stepper motor to relax the gastric band 2010. Once the pressure sensor detects pressure data within the specified pressure range, such as 4.5 and 5.5 psi, the stepper motor will stop its constriction or relaxation. To confirm the new pressure data, the caregiver can use the telemetric control unit 2070 to query the implant circuitry 2050 for pressure data. The stepper motor and the pressure sensor can then be shut off until the telemetric control unit 2070 queries the implant circuitry 2050.

The implant circuitry 2050 can also be programmed to wake up periodically and monitor the pressure data to readjust the gastric band 2010 as necessary to ensure that the pressure of the gastric band 2010 is still within the specified pressure range or other parameters. Any changes in the parameters or ranges for the pressure sensor or the other sensors in the gastric band 2010 can be permanently or semi-permanently recorded along with the date of the change and the delta of the change. This information can be supplied, for example, to the telemetric control unit 2070. In addition, the stepper motor in the gastric band 2010 can be preprogrammed with a serial number that can be sent to the telemetric control unit 2070.

In one embodiment, the telemetric control unit 2070 can include an LCD display and control panel to operate the telemetric control unit 2070. In addition, the telemetric control unit 2070 can include a series of menus allowing a user to program the gastric band 2010 to include important information such as the gastric band size, patient's name, implanting physician, and the date that the gastric band 2010 was implanted.

In addition, the telemetric control unit 2070 can communicate with the gastric band 2010 and/or the implant circuitry 2050 using telemetry through radio waves. For example, the globally recognized communications bands WMTS 402-405 Mhz or 27 Mhz can be used. An authentication process can also be used to ensure that the gastric band 2010 cannot be accidentally accessed or controlled by another control mechanism aside from the telemetric control unit 2070. The telemetric control unit 2070 can communicate with the gastric band 2010 and receive, for example, pressure data without requiring the patient to disrobe. The telemetric control unit 2070 can also be password controlled to prevent unauthorized personnel from using the device.

An exemplary pressure monitoring mechanism is described in U.S. Publication No. 2007/0156013, to the present inventor Birk, which is hereby incorporated by reference for all purposes in its entirety. However, a pressure monitoring mechanism in accordance with the present invention should not be limited to what is disclosed in the foregoing publication. Instead, a pressure monitoring mechanism in accordance with the present invention should be broadly construed as any configuration designed or implemented to monitor the pressure exerted by the stomach on the fixed-volume compartment(s) or the mechanical adjustment mechanism 2020.

An implantable banding system in accordance with exemplary embodiments of the present invention optionally comprises a self-adjusting mechanism, in turn comprising a sensor.

In accordance with exemplary embodiments, the sensor is a pressure sensor for obtaining a first pressure reading within the at least one inner fluid compartment at a first time and a second pressure reading within the at least one inner fluid compartment at a second time, and determining whether to automatically activate the mechanical adjustment mechanism based on the first pressure reading and the second pressure reading.

In accordance with other exemplary embodiments, the sensor is a pressure sensor for obtaining a minimum pressure reading within the at least one inner fluid compartment over a predetermined period of time and a maximum pressure reading within the at least one inner fluid compartment over the predetermined period of time, and determining whether to automatically activate the mechanical adjustment mechanism based on the minimum pressure reading and the maximum pressure reading. In accordance with one aspect of exemplary embodiments, the sensor calculates a standard deviation using the minimum pressure reading and the maximum pressure reading.

In accordance with yet other exemplary embodiments, the sensor is a linear motion sensor for determining a change in length of the gastric band between a first time and a second time, and determining whether to automatically activate the mechanical adjustment mechanism based on the change in the length of the band. In accordance with one aspect of exemplary embodiments, the sensor converts the length into a diameter.

An exemplary self-adjusting mechanism is described in U.S. Publication No. 2007/0156013, to the present inventor Birk, which is hereby incorporated by reference for all purposes in its entirety. However, a self-adjusting mechanism in accordance with the present invention should not be limited to what is disclosed in the foregoing publication. Instead, a self-adjusting mechanism in accordance with the present invention should be broadly construed as any configuration designed or implemented to adjust the gastric band 2010 through a variety of diameters based on information received from the pressure monitoring mechanism.

An implantable banding system in accordance with exemplary embodiments of the present invention optionally comprises a drug delivery mechanism. In exemplary embodiments, a drug is injected into the override mechanism port 2090 for later release through the membrane of the inflatable compartment 2030. In accordance with an aspect of an exemplary embodiment, the shell of the inflatable compartment 2030 is materially and/or structurally configured to optimize its function as a drug delivery membrane. In accordance with an aspect of another exemplary embodiment, the shell of the inflatable compartment 2030 is covered with a drug eluting coating for slow release of a bioactive agent.

In exemplary embodiments, the implantable banding system of the present invention is implanted by a conventional laparoscopic procedure. In exemplary embodiments, the physician first dissects the tissues around the stomach to create a tunnel for the gastric band 2010. The gastric band 2010 is then introduced into the patient's abdomen, either through an 18 mm trocar or directly through the trocar hole in the skin. The gastric band 2010 is then tunneled in place and positioned around the stomach. Finally, the antenna 2040 and the low profile port 2090 are placed just below the skin on top of the sternum.

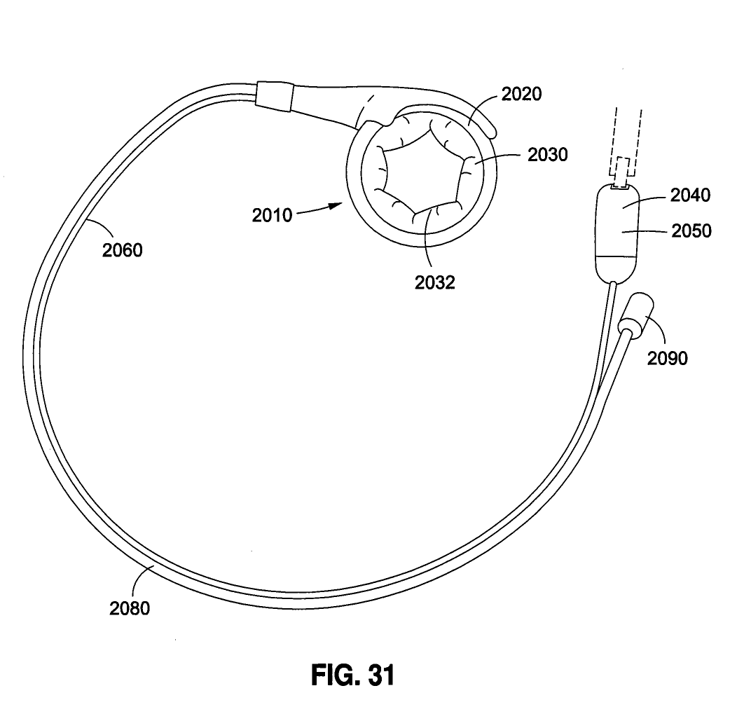

Turning finally to FIG. 31, a working embodiment of an implantable banding system in accordance with the present invention is shown.

As stated elsewhere herein, the system of the present invention has numerous applications apart from gastric banding. For example, the system of the present invention may be used for the treatment of fecal incontinence, ileostomy, coleostomy, gastro-esophageal reflux disease, urinary incontinence and isolated-organ perfusion.

For treatment of fecal incontinence, the ring may be used with little or no modifications. In addition, because the ring adjustment procedure will be performed by the patient on at least a daily basis, a portable user-friendly external control may be used. In addition, because the ring will regularly be transitioned between the closed and fully opened position, the patient microchip card is unneeded. Instead, the fully closed position may be stored in the memory of the implantable controller, and read by the external remote at each use (subject to periodic change by the physician).

A similarly modified device could be used by patients who have undergone ileostomy or coleostomy, or disposed surrounding the esophageal junction, to treat gastro-esophageal reflux disease.

For treatment of urinary incontinence, the system of the present invention may be further modified to minimize the volume of the loop surrounding the urethra by moving the actuator motor to a location elsewhere in the lower abdomen or pelvis, and coupling the actuator to the motor via a transmission cable.

The present invention also may be beneficially employed to perform isolated-organ perfusion. The treatment of certain cancers requires exposure to levels of chemotherapy agents that are too high for systemic circulation. It has been suggested that one solution to this problem is perform an open surgery procedure in which blood flow to the cancerous organ is stopped and quiescent blood replaced by circulation from an external source containing a desired dose of drug. Individual or multiple rings of the present invention may be used as valves to isolate the cancerous organ and permit perfusion of the organ with high doses of drugs. Such procedures could thus be performed on a repetitive basis without surgery, thereby reducing the trauma and the risk to the patient while improving patient outcomes.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. An implantable banding system for treating obesity, the implantable banding system comprising:
a gastric band having at least one inner fluid compartment and an outer mechanical adjustment mechanism, the at least one inner fluid compartment being filled with a volume of fluid, and the outer mechanical adjustment mechanism comprising a device configured to adjust the gastric band through a variety of diameters.

2. The implantable banding system of claim 1 wherein the volume of fluid is a fixed volume of fluid.

3. The implantable banding system of claim 1 including a sensor positioned within the at least one inner fluid compartment, configured to monitor a parameter of the fixed volume of fluid, and generate data based on the parameter to be monitored.

4. The implantable banding system of claim 3 including a telemetric control unit configured to receive data based on the parameter to be monitored.

5. The implantable banding system of claim 4 wherein the telemetric control unit further comprises a display for graphically displaying the parameter and the one or more parameter control limits for a period of time.

6. The implantable banding system of claim 4 wherein the telemetric control unit transmits a remote telemetric signal to the sensor, the remote telemetric signal comprising a request for the parameter and the one or more parameter control limits.

7. The implantable banding system of claim 4 including an implant circuit coupled to the device and configured to analyze the data from the sensor, and control operations of the device based on the data from the sensor including automatically activating the device based on the data from the sensor or transmit the data from the sensor to the telemetric control unit.

8. The implantable banding system of claim 3 wherein the parameter is selected from a group consisting of a pressure, a fill volume, a stress, a strain, a linear measurement, and combinations thereof.

9. The implantable banding system of claim 3 wherein the sensor is a pressure sensor for obtaining a first pressure reading within the at least one inner fluid compartment at a first time and a second pressure reading within the at least one inner fluid compartment at a second time, and the implant circuit determines whether to automatically activate the mechanical adjustment mechanism based on the first pressure reading and the second pressure reading.

10. The implantable banding system of claim 3 wherein the sensor is a pressure sensor for obtaining a minimum pressure reading within the at least one inner fluid compartment over a predetermined period of time and a maximum pressure reading within the at least one inner fluid compartment over the predetermined period of time, and the implant circuit determines whether to automatically activate the mechanical adjustment mechanism based on the minimum pressure reading and the maximum pressure reading.

11. The implantable banding system of claim 10 wherein the implant circuit calculates a standard deviation using the minimum pressure reading and the maximum pressure reading.

12. The implantable banding system of claim 3 wherein the sensor is a linear motion sensor for determining a change in length of the gastric band between a first time and a second time, and the implant circuit determines whether to automatically activate the mechanical adjustment mechanism based on the change in the length of the band.

13. The implantable banding system of claim 12 wherein the sensor converts the length into a diameter.

14. The implantable banding system of claim 1 wherein the fluid is selected from a group consisting of a drug, a saline solution, and combinations thereof.

15. The implantable banding system of claim 1 wherein the device is selected from a group consisting of a stepper motor, a pump, a valve, and combinations thereof.

16. The implantable banding system of claim 1 wherein the stepper motor is configured to rotate around a flexible screw within the gastric band to adjust the gastric band through a variety of diameters.

17. The implantable banding system of claim 1 further comprising an override mechanism coupled to the inner fluid compartment for releasing the fluid contained therein in the event of a failure of the system.

18. The implantable banding system of claim 17 wherein the failure is selected from a group consisting of an electrical failure, a mechanical failure, a power failure, a software failure, a hardware failure, and combinations thereof.

19. The implantable banding system of claim 1 further comprising a power source selected from a group consisting of a battery, a capacitor, a fuel cell, and combinations thereof.

20. The implantable banding system of claim 19 wherein the power source is recharged by one or more of motion, a chemical change, and a temperature change.

21. An implantable banding system for treating obesity, the implantable banding system comprising:
a telemetric control unit;
a gastric band having at least one inner fluid compartment and an outer mechanical adjustment mechanism, the at least one inner fluid compartment being filled with a fixed volume of fluid, and the outer mechanical adjustment mechanism comprising a device configured to adjust the gastric band through a variety of diameters;
a sensor positioned within the at least one inner fluid compartment, configured to monitor a parameter of the fixed volume of fluid, and generate data based on the parameter to be monitored; and
an implant circuit coupled to the device and configured to analyze the data from the sensor, and control operations of the device based on the data from the sensor including automatically activating the device based on the data from the sensor or transmit the data from the sensor to the telemetric control unit.

22. The implantable banding system of claim 21 further comprising an override mechanism coupled to the inner fluid compartment for releasing the fluid contained therein in the event of a failure of the system.

23. The implantable banding system of claim 22 wherein the failure is selected from a group consisting of an electrical failure, a mechanical failure, a power failure, a software failure, a hardware failure, and combinations thereof.

24. The implantable banding system of claim 21 wherein the parameter is selected from a group consisting of a pressure, a fill volume, a stress, a strain, a linear measurement, and combinations thereof.

25. The implantable banding system of claim 21 wherein the sensor is a pressure sensor for obtaining a first pressure reading within the at least one inner fluid compartment at a first time and a second pressure reading within the at least one inner fluid compartment at a second time, and the implant circuit determines whether to automatically activate the mechanical adjustment mechanism based on the first pressure reading and the second pressure reading.

26. The implantable banding system of claim 21 wherein the sensor is a pressure sensor for obtaining a minimum pressure reading within the at least one inner fluid compartment over a predetermined period of time and a maximum pressure reading within the at least one inner fluid compartment over the predetermined period of time, and the implant circuit determines whether to automatically activate the mechanical adjustment mechanism based on the minimum pressure reading and the maximum pressure reading.

27. The implantable banding system of claim 26 wherein the implant circuit calculates a standard deviation using the minimum pressure reading and the maximum pressure reading.

28. The implantable banding system of claim 21 wherein the sensor is a linear motion sensor for determining a change in length of the gastric band between a first time and a second time, and the implant circuit determines whether to automatically activate the mechanical adjustment mechanism based on the change in the length of the band.

29. The implantable banding system of claim 28 wherein the sensor converts the length into a diameter.

30. The implantable banding system of claim 21 wherein the fluid is selected from a group consisting of a drug, a saline solution, and combinations thereof.

31. The implantable banding system of claim 21 wherein the device is selected from a group consisting of a stepper motor, a pump, a valve, and combinations thereof.

32. The implantable banding system of claim 21 wherein the stepper motor is configured to rotate around a flexible screw within the gastric band to adjust the gastric band through a variety of diameters.

33. The implantable banding system of claim 21 wherein the telemetric control unit receives a telemetric signal comprising data related to the gastric band.

34. The implantable banding system of claim 23 wherein the telemetric control unit further comprises a display for graphically displaying the data for a period of time.

35. The implantable banding system of claim 21 wherein the telemetric control unit further comprises a display for graphically displaying the parameter and the one or more parameter control limits for a period of time.

36. The implantable banding system of claim 21 wherein the telemetric control unit transmits a remote telemetric signal to the sensor, the remote telemetric signal comprising a request for the parameter and the one or more parameter control limits.

37. The implantable banding system of claim 21 further comprising a power source selected from a group consisting of a battery, a capacitor, a fuel cell, and combinations thereof.

38. The implantable banding system of claim 27 wherein the power source is recharged by one or more of motion, a chemical change, and a temperature change.

39. A method for treating obesity comprising:
using a gastric band having at least one inner fluid compartment and an outer mechanical adjustment mechanism, the at least one inner fluid compartment being filled with a volume of fluid, and the outer mechanical adjustment mechanism comprising a device configured to adjust the gastric band through a variety of diameters.
